SEQUENCE LISTING (175 sequences)

SEQ ID NO: 1 - RNA, 23 nt, Homo sapiens
uacccuguagauccgaauuugug

SEQ ID NO: 2 - RNA, 23 nt, Homo sapiens
uacccuguagaaccgaauuugug

SEQ ID NO: 3 - RNA, 22 nt, Homo sapiens
cagugcaaugaugaaagggcau

SEQ ID NO: 4 - RNA, 22 nt, Homo sapiens
ucagugcaucacagaacuuugu

SEQ ID NO: 5 - RNA, 21 nt, Homo sapiens
ucagugcaugacagaacuugg

SEQ ID NO: 6 - RNA, 22 nt, Homo sapiens
uggagagaaaggcaguuccuga

SEQ ID NO: 7 - RNA, 21 nt, Homo sapiens
uucacaguggcuaaguucugc

SEQ ID NO: 8 - RNA, 22 nt, Homo sapiens
cuuucagucggauguuugcagc

SEQ ID NO: 9 - RNA, 23 nt, Homo sapiens
ucaagagcaauaacgaaaaaugu

SEQ ID NO: 10 - RNA, 22 nt, Homo sapiens
ugagguaguagauuguauaguu

SEQ ID NO: 11 - RNA, 23 nt, Homo sapiens
agcagcauuguacagggcuauga

SEQ ID NO: 12 - RNA, 22 nt, Homo sapiens
uucaccaccuucuccacccagc

SEQ ID NO: 13 - RNA, 21 nt, Homo sapiens
augaccuaugaauugacagac

SEQ ID NO: 14 - RNA, 22 nt, Homo sapiens
ugucaguuugucaaauacccca

SEQ ID NO: 15 - RNA, 21 nt, Homo sapiens
aucacauugccagggauuucc

SEQ ID NO: 16 - RNA, 23 nt, Homo sapiens
gcaaagcacacggccugcagaga

SEQ ID NO: 17 - RNA, 21 nt, Homo sapiens
gaacggcuucauacaggaguu

SEQ ID NO: 18 - RNA, 23 nt, Homo sapiens
ugagcgccucgacgacagagccg

SEQ ID NO: 19 - RNA, 23 nt, Homo sapiens
ucccuguccuccaggagcucacg

SEQ ID NO: 20 - RNA, 21 nt, Homo sapiens
uaguagaccguauagcguacg

SEQ ID NO: 21 - RNA, 23 nt, Homo sapiens
uagugcaauauugcuuauagggu

SEQ ID NO: 22 - RNA, 22 nt, Homo sapiens
gucauacacggcucuccucucu

SEQ ID NO: 23 - RNA, 22 nt, Homo sapiens
aaucguacagggucauccacuu

SEQ ID NO: 24 - RNA, 22 nt, Homo sapiens
ugaaacauacacgggaaaccuc

SEQ ID NO: 25 - RNA, 22 nt, Homo sapiens
cgucaacacuugcugguuuccu

SEQ ID NO: 26 - RNA, 22 nt, Homo sapiens
augcugacauauuuacuagagg

SEQ ID NO: 27 - RNA, 21 nt, Homo sapiens
uccgguucucagggcuccacc

SEQ ID NO: 28 - RNA, 22 nt, Homo sapiens
ugcggggcuagggcuaacagca

SEQ ID NO: 29 - RNA, 21 nt, Homo sapiens
uuaauaucggacaaccauugu

SEQ ID NO: 30 - RNA, 22 nt, Homo sapiens
aaacaaacauggugcacuucuu

SEQ ID NO: 31 - RNA, 17 nt, Homo sapiens
ucccuguucgggcgcca

SEQ ID NO: 32 - RNA, 22 nt, Homo sapiens
caucaucgucucaaaugagucu

SEQ ID NO: 33 - RNA, 22 nt, Homo sapiens
ucgugucuuguguugcagccgg

SEQ ID NO: 34 - RNA, 23 nt, Homo sapiens
cccaguguucagacuaccuguuc

SEQ ID NO: 35 - RNA, 21 nt, Homo sapiens
augaccuaugaauugacagac

SEQ ID NO: 36 - RNA, 22 nt, Homo sapiens
acuggacuuagggucagaaggc

SEQ ID NO: 37 - RNA, 22 nt, Homo sapiens
cgucccggggcugcgcgaggca

SEQ ID NO: 38 - RNA, 21 nt, Homo sapiens
cugggcucgggacgcgcggcu

SEQ ID NO: 39 - RNA, 21 nt, Homo sapiens
guuucaccauguuggucaggc

SEQ ID NO: 40 - RNA, 22 nt, Homo sapiens
caaaaaucucaauuacuuuugc

SEQ ID NO: 41 - RNA, 22 nt, Homo sapiens
aaaaguaauugcgguuuuugcc

SEQ ID NO: 42 - RNA, 21 nt, Homo sapiens
aggaggcagcgcucucaggac

SEQ ID NO: 43 - RNA, 22 nt, Homo sapiens
uuuccggcucgcgugggugugu

SEQ ID NO: 44 - RNA, 22 nt, Homo sapiens
uggaauguaaagaaguauguau

SEQ ID NO: 45 - RNA, 22 nt, Homo sapiens
cagugguuuuacccuaugguag

SEQ ID NO: 46 - RNA, 22 nt, Homo sapiens
cugguacaggccugggggacag

SEQ ID NO: 47 - RNA, 22 nt, Homo sapiens
uagcagcacguaaauauuggcg

SEQ ID NO: 48 - RNA, 21 nt, Homo sapiens
augaccuaugaauugacagac

SEQ ID NO: 49 - RNA, 22 nt, Homo sapiens
cauugcacuugucucggucuga

SEQ ID NO: 50 - RNA, 22 nt, Homo sapiens
uguaaacauccucgacuggaag

SEQ ID NO: 51 - RNA, 22 nt, Homo sapiens
uguaaacauccuugacuggaag

SEQ ID NO: 52 - RNA, 24 nt, Homo sapiens
uguggacagugagguagagggagu

SEQ ID NO: 53 - RNA, 20 nt, Homo sapiens
aggcaugggaggucagguga

SEQ ID NO: 54 - RNA, 23 nt, Homo sapiens
ucaaggccagaggucccacagca

SEQ ID NO: 55 - RNA, 22 nt, Homo sapiens
acuggacuuagggucagaaggc

SEQ ID NO: 56 - RNA, 18 nt, Homo sapiens
cagcagucccucccccug

SEQ ID NO: 57 - RNA, 17 nt, Homo sapiens
uucugagcugaggacag

SEQ ID NO: 58 - RNA, 18 nt, Homo sapiens
gaggcugaaggaagaugg

SEQ ID NO: 59 - RNA, 20 nt, Homo sapiens
aaaagcugggcugagaggcg

SEQ ID NO: 60 - RNA, 22 nt, Homo sapiens
uggcggcgguaguuaugggcuu

SEQ ID NO: 61 - RNA, 20 nt, Homo sapiens
cggggugggugaggucgggc

SEQ ID NO: 62 - RNA, 22 nt, Homo sapiens
uccauguuuccuucccccuucu

SEQ ID NO: 63 - RNA, 22 nt, Homo sapiens
aaaaguaauugugguuuuggcc

SEQ ID NO: 64 - RNA, 22 nt, Homo sapiens
caaaaaucucaauuacuuuugc

SEQ ID NO: 65 - RNA, 22 nt, Homo sapiens
ugugucacucgaugaccacugu

SEQ ID NO: 66 - RNA, 22 nt, Homo sapiens
uggucuaggauuguuggaggag

SEQ ID NO: 67 - RNA, 22 nt, Homo sapiens
guucucccaacguaagcccagc

SEQ ID NO: 68 - RNA, 22 nt, Homo sapiens
uacccauugcauaucggaguug

SEQ ID NO: 69 - RNA, 21 nt, Homo sapiens
cgcgggugcuuacugacccuu

SEQ ID NO: 70 - RNA, 21 nt, Homo sapiens
gucagcggaggaaaagaaacu

SEQ ID NO: 71 - RNA, 22 nt, Homo sapiens
auauaauacaaccugcuaagug

SEQ ID NO: 72 - RNA, 23 nt, Homo sapiens
agcagcauuguacagggcuauga

SEQ ID NO: 73 - RNA, 21 nt, Homo sapiens
uaaagugcugacagugcagau

SEQ ID NO: 74 - RNA, 22 nt, Homo sapiens
ucguaccgugaguaauaaugcg

SEQ ID NO: 75 - RNA, 21 nt, Homo sapiens
cauuauuacuuuugguacgcg

SEQ ID NO: 76 - RNA, 18 nt, Homo sapiens
gucccuguucaggcgcca

SEQ ID NO: 77 - RNA, 17 nt, Homo sapiens
ucccuguucgggcgcca

SEQ ID NO: 78 - RNA, 22 nt, Homo sapiens
uaacagucuacagccauggucg

SEQ ID NO: 79 - RNA, 21 nt, Homo sapiens
uaccacaggguagaaccacgg

SEQ ID NO: 80 - RNA, 23 nt, Homo sapiens
uguaguguuuccuacuuuaugga

SEQ ID NO: 81 - RNA, 22 nt, Homo sapiens
ugagaacugaauuccauaggcu

SEQ ID NO: 82 - RNA, 22 nt, Homo sapiens
ucucccaacccuuguaccagug

SEQ ID NO: 83 - RNA, 22 nt, Homo sapiens
uagcagcacaucaugguuuaca

SEQ ID NO: 84 - RNA, 22 nt, Homo sapiens
aacauucauugcugucgguggg

SEQ ID NO: 85 - RNA, 22 nt, Homo sapiens
caaagaauucuccuuuugggcu

SEQ ID NO: 86 - RNA, 22 nt, Homo sapiens
uagcuuaucagacugauguuga

SEQ ID NO: 87 - RNA, 21 nt, Homo sapiens
agcuacaucuggcuacugggu

SEQ ID NO: 88 - RNA, 22 nt, Homo sapiens
cguguauuugacaagcugaguu

SEQ ID NO: 89 - RNA, 22 nt, Homo sapiens
uggcucaguucagcaggaacag

SEQ ID NO: 90 - RNA, 21 nt, Homo sapiens
uucaaguaauucaggauaggu

SEQ ID NO: 91 - RNA, 22 nt, Homo sapiens
cuuucagucggauguuugcagc

SEQ ID NO: 92 - RNA, 22 nt, Homo sapiens
uguaaacauccccgacuggaag

SEQ ID NO: 93 - RNA, 22 nt, Homo sapiens
aaaagcuggguugagagggcga

SEQ ID NO: 94 - RNA, 20 nt, Homo sapiens
aaaagcuggguugagagggu

SEQ ID NO: 95 - RNA, 22 nt, Homo sapiens
cuggcccucucugcccuuccgu

SEQ ID NO: 96 - RNA, 22 nt, Homo sapiens
uuuguucguucggcucgcguga

SEQ ID NO: 97 - RNA, 22 nt, Homo sapiens
cuccugacuccagguccugugu

SEQ ID NO: 98 - RNA, 22 nt, Homo sapiens
agcucggucugaggccccucag

SEQ ID NO: 99 - RNA, 17 nt, Homo sapiens
accccacuccugguacc

SEQ ID NO: 100 - RNA, 22 nt, Homo sapiens
aaaccguuaccauuacugaguu

SEQ ID NO: 101 - RNA, 17 nt, Homo sapiens
guggaccuggcugggac

SEQ ID NO: 102 - RNA, 22 nt, Homo sapiens
aagacgggaggaaagaagggag

SEQ ID NO: 103 - RNA, 21 nt, Homo sapiens
caaagcgcuucccuuuggagc

SEQ ID NO: 104 - RNA, 22 nt, Homo sapiens
caugccuugaguguaggaccgu

SEQ ID NO: 105 - RNA, 22 nt, Homo sapiens
gagcuuauucauaaaagugcag

SEQ ID NO: 106 - RNA, 22 nt, Homo sapiens
uggucuaggauuguuggaggag

SEQ ID NO: 107 - RNA, 22 nt, Homo sapiens
uacccauugcauaucggaguug

SEQ ID NO: 108 - RNA, 22 nt, Homo sapiens
uccauuacacuacccugccucu

SEQ ID NO: 109 - RNA, 22 nt, Homo sapiens
uauugcacuugucccggccugu

SEQ ID NO: 110 - RNA, 22 nt, Homo sapiens
cacccguagaaccgaccuugcg

SEQ ID NO: 111 - RNA, 22 nt, Homo sapiens
ggagacgcggcccuguuggagu

SEQ ID NO: 112 - RNA, 22 nt, Homo sapiens
cugugcgugugacagcggcuga

SEQ ID NO: 113 - RNA, 21 nt, Homo sapiens
agggcccccccucaauccugu

SEQ ID NO: 114 - RNA, 21 nt, Homo sapiens
cggggcagcucaguacaggau

SEQ ID NO: 115 - RNA, 23 nt, Homo sapiens
uaauccuugcuaccugggugaga

SEQ ID NO: 116 - RNA, 22 nt, Homo sapiens
ugugacagauugauaacugaaa

SEQ ID NO: 117 - RNA, 23 nt, Homo sapiens
ucauagcccuguacaaugcugcu

SEQ ID NO: 118 - RNA, 22 nt, Homo sapiens
ucccugagacccuuuaaccugu

SEQ ID NO: 119 - RNA, 22 nt, Homo sapiens
cuuggcaccuagcaagcacuca

SEQ ID NO: 120 - RNA, 17 nt, Homo sapiens
ucccuguucgggcgcca

SEQ ID NO: 121 - RNA, 22 nt, Homo sapiens
gcuacuucacaacaccagggcc

SEQ ID NO: 122 - RNA, 22 nt, Homo sapiens
ugcccuguggacucaguucugg

SEQ ID NO: 123 - RNA, 22 nt, Homo sapiens
ugauauguuugauauauuaggu

SEQ ID NO: 124 - RNA, 23 nt, Homo sapiens
cccaguguucagacuaccuguuc

SEQ ID NO: 125 - RNA, 22 nt, Homo sapiens
ccuguucuccauuacuuggcuc

SEQ ID NO: 126 - RNA, 20 nt, Homo sapiens
aaaagcuggguugagagggu

SEQ ID NO: 127 - RNA, 22 nt, Homo sapiens
aaagacauaguugcaagauggg

SEQ ID NO: 128 - RNA, 24 nt, Homo sapiens
aauccuuggaaccuaggugugagu

SEQ ID NO: 129 - RNA, 23 nt, Homo sapiens
ugagcaccacacaggccgggcgc

SEQ ID NO: 130 - RNA, 22 nt, Homo sapiens
aauugcacuuuagcaaugguga

SEQ ID NO: 131 - RNA, 23 nt, Homo sapiens
ucaaggccagaggucccacagca

SEQ ID NO: 132 - RNA, 22 nt, Homo sapiens
cagcagcaauucauguuuugaa

SEQ ID NO: 133 - RNA, 23 nt, Homo sapiens
ucuuggaguaggucauugggugg

SEQ ID NO: 134 - RNA, 20 nt, Homo sapiens
aaaagcugggcugagaggcg

SEQ ID NO: 135 - RNA, 23 nt, Homo sapiens
ucaaaauguagaggaagacccca

SEQ ID NO: 136 - RNA, 22 nt, Homo sapiens
acccagguucccucuggccgca

SEQ ID NO: 137 - RNA, 23 nt, Homo sapiens
uggccggaugggacaggaggcau

SEQ ID NO: 138 - RNA, 21 nt, Homo sapiens
cagcagcacacugugguuugu

SEQ ID NO: 139 - RNA, 22 nt, Homo sapiens
aaugcaccugggcaaggauuca

SEQ ID NO: 140 - RNA, 22 nt, Homo sapiens
aaagugcuucucuuuggugggu

SEQ ID NO: 141 - RNA, 22 nt, Homo sapiens
aagugcuuccuuuuagaggguu

SEQ ID NO: 142 - RNA, 22 nt, Homo sapiens
ugugucacucgaugaccacugu

SEQ ID NO: 143 - RNA, 22 nt, Homo sapiens
gacuauagaacuuucccccuca

SEQ ID NO: 144 - RNA, 22 nt, Homo sapiens
augcugacauauuuacuagagg

SEQ ID NO: 145 - RNA, 21 nt, Homo sapiens
uggguuuacguugggagaacu

SEQ ID NO: 146 - RNA, 22 nt, Homo sapiens
uuugugaccugguccacuaacc

SEQ ID NO: 147 - RNA, 22 nt, Homo sapiens
auauaauacaaccugcuaagug

SEQ ID NO: 148 - RNA, 22 nt, Homo sapiens
ugagguaggagguuguauaguu

SEQ ID NO: 149 - RNA, 22 nt, Homo sapiens
ggagacgcggcccuguuggagu

SEQ ID NO: 150 - RNA, 22 nt, Homo sapiens
uaacacugucugguaaagaugg

SEQ ID NO: 151 - RNA, 22 nt, Homo sapiens
ugcccuguggacucaguucugg

SEQ ID NO: 152 - RNA, 22 nt, Homo sapiens
aacauucaaccugucggugagu

SEQ ID NO: 153 - RNA, 22 nt, Homo sapiens
aaagaucuggaagugggagaca

SEQ ID NO: 154 - RNA, 24 nt, Homo sapiens
aauccuuggaaccuaggugugagu

SEQ ID NO: 155 - RNA, 21 nt, Homo sapiens
uaguagaccguauagcguacg

SEQ ID NO: 156 - RNA, 22 nt, Homo sapiens
acuggacuuagggucagaaggc

SEQ ID NO: 157 - RNA, 17 nt, Homo sapiens
guggaccuggcugggac

SEQ ID NO: 158 - RNA, 22 nt, Homo sapiens
gucauacacggcucuccucucu

SEQ ID NO: 159 - RNA, 21 nt, Homo sapiens
cggggcagcucaguacaggau

SEQ ID NO: 160 - RNA, 22 nt, Homo sapiens
aaucguacagggucauccacuu

SEQ ID NO: 161 - RNA, 22 nt, Homo sapiens
ugaaacauacacgggaaaccuc

SEQ ID NO: 162 - RNA, 21 nt, Homo sapiens
gugucuuuugcucugcaguca

SEQ ID NO: 163 - RNA, 22 nt, Homo sapiens
aagugcuuccuuuuagaggguu

SEQ ID NO: 164 - RNA, 22 nt, Homo sapiens
caaaaaucucaauuacuuuugc

SEQ ID NO: 165 - RNA, 22 nt, Homo sapiens
aaaaguaauugcgguuuuugcc

SEQ ID NO: 166 - RNA, 22 nt, Homo sapiens
gagcuuauucauaaaagugcag

SEQ ID NO: 167 - RNA, 21 nt, Homo sapiens
uccgguucucagggcuccacc

SEQ ID NO: 168 - RNA, 21 nt, Homo sapiens
cgcgggugcuuacugacccuu

SEQ ID NO: 169 - RNA, 21 nt, Homo sapiens
uuaauaucggacaaccauugu

SEQ ID NO: 170 - RNA, 22 nt, Homo sapiens
uauugcacuugucccggccugu

SEQ ID NO: 171 - RNA, 21 nt, Homo sapiens
cugaccuaugaauugacagcc

SEQ ID NO: 172 - RNA, 22 nt, Homo sapiens
uagcaccaucugaaaucgguua

SEQ ID NO: 173 - RNA, 23 nt, Homo sapiens
uguaaacauccuacacucucagc

SEQ ID NO: 174 - RNA, 22 nt, Homo sapiens
aagacgggaggaaagaagggag

SEQ ID NO: 175 - RNA, 22 nt, Homo sapiens
auauaauacaaccugcuaagug

(19) United States
(12) Patent Application Publication
(10) Pub. No.: US 2018/0251837 A1
(43) Pub. Date: Sep. 6, 2018

(54) USE OF MIRCO-RNAS CIRCULATING IN THE BLOOD SERUM OR BLOOD PLASMA FOR IDENTIFYING PATIENTS REQUIRING A BIOPSY AND AS A MARKER FOR THE DIFFERENTIAL DIAGNOSIS OF INDIVIDUAL NON-ISCHEMIC CARDIOMYOPATHIES OR STORAGE DISEASES

(71) Applicant: IKDT INSTITUT KARDIALE DIAGNOSTIK UND THERAPIE GMBH, Berlin (DE)

(72) Inventors: Heinz-Peter SCHULTHEISS, Berlin (DE); Dirk LASSNER, Stahnsdorf (DE); Maria ROHDE, Berlin (DE); Christine SIEGISMUND, Berlin (DE); Max KOHLENBERG, Berlin (DE)

(73) Assignee: IKDT INSTITUT KARDIALE DIAGNOSTIK UND THERAPIE GMBH, Berlin (DE)

(21) Appl. No.: 15/756,908

(22) PCT Filed: Sep. 1, 2016
(86) PCT No.: PCT/EP2016/070643

§ 371 Date: Mar. 1, 2018

(30) Foreign Application Priority Data
Sep. 2, 2015 (DE) 10 2015 216 782.8

(51) Int. Cl. C12Q 1/6883; C12Q 1/70

(52) U.S. Cl. CPC C12Q 1/6883; C12Q 1/70; C12Q 2600/178; C12Q 2600/158; C12Q 2600/112

(57) ABSTRACT

The invention relates to the use of certain micro-RNAs circulating in the blood serum or blood plasma in the form of diagnostic profiles as a marker in an in vitro method for distinguishing between patients for which a myocardial biopsy is necessary for the diagnostic identification of a heart muscle disease and patients for which myocardial biopsy is not necessary, and for the optional differential diagnosis of individual non-ischemic cardiomyopathies or storage diseases affecting the heart.

### CROSS-REFERENCE TO A RELATED APPLICATION

This application is a national phase patent application of International patent application PCT/EP2016/070643, filed on Sep. 1, 2016, which claims priority of German patent application 10 2015 216 782.8, filed on Sep. 2, 2015.

### BACKGROUND

In an aspect, the invention relates to the use of certain microRNAs circulating in blood serum/blood plasma in a method for differentiating between patients requiring, for the diagnostic identification of cardiomyopathy, myocardial biopsy, and patients not requiring myocardial biopsy, and for the optional differential diagnostic differentiation between individual non-ischemic cardiomyopathies or storage diseases of the heart. Further aspects of the invention relate to a diagnostic system for the same purpose and to a medicament comprising specific microRNAs.

Today, in western countries, cardiovascular disorders are by far the most frequent cause of death. The incidence of heart failure in Europe and the USA is 15 and 12 million cases, respectively. At about 5 million cases (30%), in Europe dilated cardiomyopathy (DCM) is the most frequent form of non-ischemic cardiomyopathy. Without special treatment, the 5-year survival rate of this viral/inflammatory-induced heart disease is 50%. Furthermore, in 45% of all patients requiring a transplantation, this serious heart damage developed on the basis of an existing DCM. The high incidence of non-ischemic cardiomyopathy and the enormous health-economical consequences of these diseases require early and specific diagnosis and, based on this, a targeted therapy. This equally applies to storage diseases of the heart, such as cardiomyopathies caused by amyloidosis.

Cardiomyopathy is understood to mean disorders of the myocardium which are primarily not the sequela of other disorders of the cardiovascular system. Thus, these disorders are neither the result of mechanical overload (e.g. owing to high blood pressure or a valve defect) nor the result of ischemia of the coronary vessels (coronary heart disease).

In contrast to coronary heart diseases, where there are many diagnosis options, hitherto there have been no specific non-invasive diagnosis parameters for the different forms of non-ischemic cardiomyopathies and storage diseases of the heart. Hitherto, owing to their multifarious etiologies, non-ischemic cardiomyopathies and storage diseases of the heart could only be diagnosed accurately by using invasive methods (myocardial biopsy), with the aim of achieving further therapy-relevant differentiation of the individual pathological states.

Currently, it is assumed that, in addition to the purely genetic forms, cardiomyopathies are frequently caused by a virus infection and/or an inflammatory reaction associated therewith, where genetic predispositions may be relevant for the progression of the disease. To date, pathogenetically, these overlapping pathological states have been insufficiently elucidated. For this reason, it is important to develop a diagnostic methodology which allows early diagnosis of the different forms of clinically indistinguishable groups of non-ischemic cardiomyopathies and storage diseases of the heart to allow initiation of a therapy appropriate for the patient at the earliest possible time.

The gold standard of cardiomyopathy diagnosis is myocardial biopsy; however, this is only performed at special cardiology centers and in a few countries, and even there on only a highly restricted number of patients. In addition, the cardiac sample is frequently only examined histologically, without the immunohistochemical inflammation differentiation and the molecular biological examinations for viral infections required.

However, considering the prevalence of non-ischemic cardiomyopathies and storage diseases of the heart and the enormous health-economical consequences, early identification, using minimally-invasive diagnostic methods, of patients who are suspected to suffer from viral inflammatory cardiomyopathy would be desirable. In order to prevent irreversible myocardial damage, a myocardial biopsy should be taken from these patients as quickly as possible so that, if required, they can then be subjected to a specific therapy.

Furthermore, it is important to know which patient responds to which therapy. Initial studies indicate that the presence of individual gene expression patterns is possibly associated with a genetic predisposition which may affect the individually required and effective treatment of the patient.

By now, microRNAs (miRNAs) have been recognized as important regulators of genetic expression, including their significance in the etiology of myocardium disorders. MicroRNAs are single-stranded short ribonucleic acid molecules (RNA molecules) of a length of 17 to 24 bases which are involved directly in gene regulation. Here, in particular, degradation of the target RNA or translation are controlled. MicroRNAs form a novel regulatory cycle of disease-related and tissue-specific gene expression. Increasingly, microRNAs circulating in human serum are used as stable biomarkers for the diagnosis of various diseases and for monitoring therapies applied.

In the publication “MicroRNA signatures in peripheral blood mononuclear cells of chronic heart failure patients”, Physiol. Genomics 42 (2010), pages 420-426, Voellenkle et al. describe various microRNAs used for differentiating between healthy individuals and patients suffering from ischemic cardiomyopathy (ICM) or non-ischemic dilated cardiomyopathy (NIDCM). However, based on the microRNAs identified, it was not possible to differentiate between patients suffering from ischemic cardiomyopathy and patients suffering from non-ischemic dilated cardiomyopathy.

In the publication “Altered microRNA expression in human heart disease”, Physiol. Genomics 31 (2007), pages 367-373, Ikeda et al. likewise describe various microRNAs which were used to differentiate between different cardiac disorders, i.e. ischemic cardiomyopathy (ICM), dilated cardiomyopathy (DCM) and aortic stenosis (AS; as a valve defect, this disorder does not fall under cardiomyopathies).

In the publication “Unique microRNA profile in end-stage heart failure indicates alterations in specific cardiovascular signaling networks”, J. Biol. Chem. 284 (2009), pages 27487-27499, Prasad et al. identify eight microRNAs whose tissue concentration is changed in the event of heart failure. Here, however, only patients suffering from final-stage dilated cardiomyopathy were examined. Differentiation of different myocardial disorders from one another was not the aim of this publication and, accordingly, is not described.

In the publication “Differential Cardiac microRNA expression predicts the clinical course in human enterovirus cardiomyopathy”, Circ. Heart Failure 8(3) (2015), pages 608-615, Kühl et al. identify 16 microRNAs whose tissue concentration is predictive of a spontaneous elimination or long-term persistence of a myocardial enterovirus infection. Persistence correlates with a significantly worse prognosis and is an indication for treatment with interferon-beta. These investigations demonstrate the diagnostic-therapeutic potential of the microRNAs. Such studies have hitherto only been shown for microRNAs from myocardial biopsies.

WO 2013/127782 A2 discloses the use of specific nucleic acids as markers for identifying individual forms of non-ischemic cardiomyopathies or storage diseases of the heart. In that international patent application, primarily myocardial biopsies and peripheral blood cells were studied.

Also frequent are infections of the myocardium which initially do not result in cardiomyopathy. Thus, clinical findings show a heart of normal size and normal performance infected, for example, by a virus. This infection may subsequently lead to cardiomyopathy; accordingly, it is a precursor of cardiomyopathy. If diagnosed and treated early, however, the onset of cardiomyopathy can be avoided.

Hitherto, attempts to make microRNA-based diagnoses using heart tissue gene profiles, which diagnoses allow the identification and differentiation of different types of non-ischemic cardiomyopathies, storage diseases of the heart and myocardium infections without cardiomyopathic symptoms, have been successful (cf., for example, WO 2013/127782 A2).

### SUMMARY

The object of aspects of the present invention is to provide microRNAs suitable for appropriate identification of patients suspected of suffering from non-ischemic cardiomyopathy, storage diseases of the heart or infections of the myocardium without cardiomyopathic symptoms and to provide a corresponding diagnostic system based thereon, where the detection of the corresponding microRNAs should be possible by a method less invasive than the analysis of a myocardial biopsy.

This object is achieved by an in-vitro method to differentiate between patients who, for diagnostic identification of a myocardial disorder, require myocardial biopsy, and patients not requiring myocardial biopsy, and for the optional differential diagnostic differentiation of individual non-ischemic cardiomyopathies or storage diseases of the heart.

The method serves in particular to provide initial data for subsequent diagnosis or diagnostic classification.

In this method, a blood serum or blood plasma sample (hereinbelow referred to in short as blood serum/blood plasma sample) (optionally pre-processed) of a patient is analyzed using a diagnostic profile. The diagnostic profile comprises at least 2 microRNAs or synthetic nucleic acids having identical or complementary sequences, selected from the group consisting of the following microRNAs: hsa-let-7f-5p (SEQ ID NO: 10), hsa-miR-103a-3p (SEQ ID NO: 11), hsa-miR-197-3p (SEQ ID NO: 12), hsa-miR-215-5p (SEQ ID NO: 13), hsa-miR-223-3p (SEQ ID NO: 14), hsa-miR-23a-3p (SEQ ID NO: 15), hsa-miR-330-3p (SEQ ID NO: 16), hsa-miR-337-5p (SEQ ID NO: 17), hsa-miR-339-3p (SEQ ID NO: 18), hsa-miR-339-5p (SEQ ID NO: 19), hsa-miR-411-5p (SEQ ID NO: 20), hsa-miR-454-3p (SEQ ID NO: 21), hsa-miR-485-3p (SEQ ID NO: 22), hsa-miR-487b-3p (SEQ ID NO: 23), hsa-miR-494-3p (SEQ ID NO: 24), hsa-miR-505-3p (SEQ ID NO: 25), hsa-miR-628-5p (SEQ ID NO: 26), hsa-miR-671-3p (SEQ ID NO: 27), hsa-miR-744-5p (SEQ ID NO: 28), hsa-miR-889-3p (SEQ ID NO: 29), mmu-miR-495-3p (SEQ ID NO: 30).

Accordingly, a plurality (at least 2) of defined microRNAs are used as markers for identifying patients requiring biopsy.

A sequence is considered to be “identical” if it corresponds at least 90%, in particular at least 95%, in particular at least 97%, in particular at least 98%, in particular at least 99%, in particular at least 99.5% and very particularly at least 99.9% to the respective sequence used for comparison.

The microRNA names used above are unambiguous names, generally known to the person skilled in the art, for different microRNA sequences. The respective sequences behind these names can be retrieved, for example, from the freely accessible (under the internet address http://www.mirbase.org) database miRBase; in addition, they are listed in the appended sequence protocol.

If specific names of those mentioned above are used both for a stem-loop structure and for a mature microRNA sequence, the respective abbreviation refers in each case to the mature microRNA sequence.

Aspects of the claimed invention are based on the concept of using a diagnostic profile of microRNAs as marker in order to initially differentiate between healthy (no biopsy required) patients and patients suffering from cardiomyopathy and optionally to confirm the presence of a subtype of a non-ischemic cardiomyopathy or a storage disease of the heart. The primary object of aspects of the present invention is to differentiate between healthy patients and sick patients. In a second step, patients requiring biopsy can be classified into the specific sub-groups of non-ischemic cardiomyopathies and sub-groups of storage diseases of the heart.

As illustrated, the microRNAs of the above list and all the lists that follow are employed in the form of diagnostically relevant profiles. Such a profile may consist, for example, of at least or exactly 2, 3, 4, 5, 6, 7, 8, 9, 10, 11, 12, 13, 14, 15, 20, 30, 40, 50 or else all microRNAs of the above list or the respective lists that follow.

In one variant, the diagnostic profile additionally comprises at least 3 microRNAs or synthetic nucleic acids having identical or complementary sequences, selected from the group consisting of the following microRNAs: hsa-miR-1274B (SEQ ID NO: 31), hsa-miR-136-3p (SEQ ID NO: 32), hsa-miR-187-3p (SEQ ID NO: 33), hsa-miR-199a-5p (SEQ ID NO: 34), hsa-miR-215-5p (SEQ ID NO: 35), hsa-miR-422a (SEQ ID NO: 36), hsa-miR-4449 (SEQ ID NO: 37), hsa-miR-4674 (SEQ ID NO: 38), hsa-miR-5096 (SEQ ID NO: 39), hsa-miR-548c-3p (SEQ ID NO: 40), hsa-miR-548c-5p (SEQ ID NO: 41), hsa-miR-650 (SEQ ID NO: 42). In this case, the diagnostic profile is also suitable for differential diagnosis or for identifying adenovirus-induced cardiomyopathy.

In one variant, the diagnostic profile additionally comprises at least 3 microRNAs or synthetic nucleic acids having identical or complementary sequences, selected from the group consisting of the following microRNAs: hsa-miR-1180-3p (SEQ ID NO: 43), hsa-miR-1-3p (SEQ ID NO: 44), hsa-miR-140-5p (SEQ ID NO: 45), hsa-miR-150-3p (SEQ ID NO: 46), hsa-miR-16-5p (SEQ ID NO: 47), hsa-miR-215-5p (SEQ ID NO: 48), hsa-miR-25-3p (SEQ ID NO: 49), hsa-miR-30a-5p (SEQ ID NO: 50), hsa-miR-30e-5p (SEQ ID NO: 51), hsa-miR-3138 (SEQ ID NO: 52), hsa-miR-3622b-5p (SEQ ID NO: 53), hsa-miR-3922-5p (SEQ ID NO: 54), hsa-miR-422a (SEQ ID NO: 55), hsa-miR-4274 (SEQ ID NO: 56), hsa-miR-4303 (SEQ ID NO: 57), hsa-miR-4419b (SEQ ID NO: 58), hsa-miR-4429 (SEQ ID NO: 59), hsa-miR-4467 (SEQ ID NO: 60), hsa-miR-4651 (SEQ ID NO: 61), hsa-miR-4716-5p (SEQ ID NO: 62), hsa-miR-548b-5p (SEQ ID NO: 63), hsa-miR-548c-3p (SEQ ID NO: 64), hsa-miR-597-5p (SEQ ID NO: 65), hsa-miR-601 (SEQ ID NO: 66), hsa-miR-629-3p (SEQ ID NO: 67), hsa-miR-660-5p (SEQ ID NO: 68), hsa-miR-886-3p (SEQ ID NO: 69), hsa-miR-923 (SEQ ID NO: 70), mmu-miR-374-5p (SEQ ID NO: 71). In this case, the diagnostic profile is also suitable for differential diagnosis or for identifying enterovirus-(coxsackievirus-)induced cardiomyopathy.

In one variant, the diagnostic profile for identifying an amyloidosis of the heart additionally comprises at least 3 microRNAs or synthetic nucleic acids having identical or complementary sequences, selected from the group consisting of the following microRNAs: hsa-miR-103a-3p (SEQ ID NO: 72), hsa-miR-106b-5p (SEQ ID NO: 73), hsa-miR-126-3p (SEQ ID NO: 74), hsa-miR-126-5p (SEQ ID NO: 75), hsa-miR-1274A (SEQ ID NO: 76), hsa-miR-1274B (SEQ ID NO: 77), hsa-miR-132-3p (SEQ ID NO: 78), hsa-miR-140-3p (SEQ ID NO: 79), hsa-miR-142-3p (SEQ ID NO: 80), hsa-miR-146b-5p (SEQ ID NO: 81), hsa-miR-150-5p (SEQ ID NO: 82), hsa-miR-15b-5p (SEQ ID NO: 83), hsa-miR-181b-5p (SEQ ID NO: 84), hsa-miR-186-5p (SEQ ID NO: 85), hsa-miR-21-5p (SEQ ID NO: 86), hsa-miR-222-3p (SEQ ID NO: 87), hsa-miR-223-5p (SEQ ID NO: 88), hsa-miR-24-3p (SEQ ID NO: 89), hsa-miR-26b-5p (SEQ ID NO: 90), hsa-miR-30a-3p (SEQ ID NO: 91), hsa-miR-30d-5p (SEQ ID NO: 92), hsa-miR-320a (SEQ ID NO: 93), hsa-miR-320c (SEQ ID NO: 94), hsa-miR-328-3p (SEQ ID NO: 95), hsa-miR-375 (SEQ ID NO: 96), hsa-miR-378-5p (SEQ ID NO: 97), hsa-mir-423 (SEQ ID NO: 98), hsa-miR-4286 (SEQ ID NO: 99), hsa-miR-451a (SEQ ID NO: 100), hsa-miR-4535 (SEQ ID NO: 101), hsa-miR-483-5p (SEQ ID NO: 102), hsa-miR-518d-3p (SEQ ID NO: 103), hsa-miR-532-5p (SEQ ID NO: 104), hsa-miR-590-5p (SEQ ID NO: 105), hsa-miR-601 (SEQ ID NO: 106), hsa-miR-660-5p (SEQ ID NO: 107), hsa-miR-885-5p (SEQ ID NO: 108), hsa-miR-92a-3p (SEQ ID NO: 109), hsa-miR-99b-5p (SEQ ID NO: 110). In this case, the diagnostic profile is also suitable for differential diagnosis or for identifying amyloidosis of the heart.

In a further variant, such an amyloidosis may be subdivided more specifically and accordingly be diagnosed more accurately. Thus, a subclassification for the storage disease amyloidosis may be carried out which allows differentiation between ATTR amyloidosis and lambda amyloidosis.

Thus, the following microRNAs are suitable for identifying lambda amyloidosis of the heart: hsa-miR-106b-5p (SEQ ID NO: 73), hsa-miR-1274A (SEQ ID NO: 76), hsa-miR-30a-3p (SEQ ID NO: 91), hsa-miR-4535 (SEQ ID NO: 101). In one variant, the diagnostic profile thus additionally comprises at least 3 microRNAs or synthetic nucleic acids having identical or complementary sequences, selected from the group consisting of the microRNAs mentioned above.

The following microRNAs are suitable for identifying an ATTR amyloidosis of the heart: hsa-miR-140-3p (SEQ ID NO: 79), hsa-miR-181b-5p (SEQ ID NO: 84), hsa-miR-222-3p (SEQ ID NO: 87), hsa-miR-223-5p (SEQ ID NO: 88). In one variant, the diagnostic profile thus additionally comprises at least 3 microRNAs or synthetic nucleic acids having identical or complementary sequences, selected from the group consisting of the microRNAs mentioned above.

In one variant, the diagnostic profile additionally comprises at least 3 microRNAs or synthetic nucleic acids having identical or complementary sequences, selected from the group consisting of the following microRNAs: hsa-miR-139-3p (SEQ ID NO: 111), hsa-miR-210-3p (SEQ ID NO: 112), hsa-miR-296-5p (SEQ ID NO: 113), hsa-miR-486-3p (SEQ ID NO: 114), hsa-miR-500a-5p (SEQ ID NO: 115), hsa-miR-542-3p (SEQ ID NO: 116). In this case, the diagnostic profile is also suitable for differential diagnosis or for identifying cardiac sarcoidosis/giant cell myocarditis of the heart.

In one variant, the diagnostic profile additionally comprises at least 3 microRNAs or synthetic nucleic acids having identical or complementary sequences, selected from the group consisting of the following microRNAs: hsa-miR-103b (SEQ ID NO: 117), hsa-miR-125a-5p (SEQ ID NO: 118), hsa-miR-1271-5p (SEQ ID NO: 119), hsa-miR-1274B (SEQ ID NO: 120), hsa-miR-138-1-3p (SEQ ID NO: 121), hsa-miR-146b-3p (SEQ ID NO: 122), hsa-miR-190a-5p (SEQ ID NO: 123), hsa-miR-199a-5p (SEQ ID NO: 124), hsa-miR-26b-3p (SEQ ID NO: 125), hsa-miR-320c (SEQ ID NO: 126), hsa-miR-3617-5p (SEQ ID NO: 127), hsa-miR-362-5p (SEQ ID NO: 128), hsa-miR-3663-3p (SEQ ID NO: 129), hsa-miR-367-3p (SEQ ID NO: 130), hsa-miR-3922-5p (SEQ ID NO: 131), hsa-miR-424-5p (SEQ ID NO: 132), hsa-miR-432-5p (SEQ ID NO: 133), hsa-miR-4429 (SEQ ID NO: 134), hsa-miR-4698 (SEQ ID NO: 135), hsa-miR-4726-3p (SEQ ID NO: 136), hsa-miR-4743-5p (SEQ ID NO: 137), hsa-miR-497-5p (SEQ ID NO: 138), hsa-miR-502-3p (SEQ ID NO: 139), hsa-miR-520d-3p (SEQ ID NO: 140), hsa-miR-520f-3p (SEQ ID NO: 141), hsa-miR-597-5p (SEQ ID NO: 142), hsa-miR-625-3p (SEQ ID NO: 143), hsa-miR-628-5p (SEQ ID NO: 144), hsa-miR-629-5p (SEQ ID NO: 145), hsa-miR-758-3p (SEQ ID NO: 146), mmu-miR-374-5p (SEQ ID NO: 147). In this case, the diagnostic profile is also suitable for differential diagnosis or for identifying erythrovirus-induced cardiomyopathy.

In one variant, the diagnostic profile additionally comprises at least 3 microRNAs or synthetic nucleic acids having identical or complementary sequences, selected from the group consisting of the following microRNAs: hsa-let-7e-5p (SEQ ID NO: 148), hsa-miR-139-3p (SEQ ID NO: 149), hsa-miR-141-3p (SEQ ID NO: 150), hsa-miR-146b-3p (SEQ ID NO: 151), hsa-miR-181c-5p (SEQ ID NO: 152), hsa-miR-3156-5p (SEQ ID NO: 153), hsa-miR-362-5p (SEQ ID NO: 154), hsa-miR-411-5p (SEQ ID NO: 155), hsa-miR-422a (SEQ ID NO: 156), hsa-miR-4535 (SEQ ID NO: 157), hsa-miR-485-3p (SEQ ID NO: 158), hsa-miR-486-3p (SEQ ID NO: 159), hsa-miR-487b-3p (SEQ ID NO: 160), hsa-miR-494-3p (SEQ ID NO: 161), hsa-miR-511-5p (SEQ ID NO: 162), hsa-miR-520f-3p (SEQ ID NO: 163), hsa-miR-548c-3p (SEQ ID NO: 164), hsa-miR-548c-5p (SEQ ID NO: 165), hsa-miR-590-5p (SEQ ID NO: 166), hsa-miR-671-3p (SEQ ID NO: 167), hsa-miR-886-3p (SEQ ID NO: 168), hsa-miR-889-3p (SEQ ID NO: 169), hsa-miR-92a-3p (SEQ ID NO: 170). In this case, the diagnostic profile is also suitable for differential diagnosis or for identifying ciHHV6-induced cardiomyopathy.

In one variant, the diagnostic profile additionally comprises at least 3 microRNAs or synthetic nucleic acids having identical or complementary sequences, selected from the group consisting of the following microRNAs: hsa-miR-192-5p (SEQ ID NO: 171), hsa-miR-29a-3p (SEQ ID NO: 172), hsa-miR-30c-5p (SEQ ID NO: 173), hsa-miR-483-5p (SEQ ID NO: 174), mmu-miR-374-5p (SEQ ID NO: 175). In this case, the diagnostic profile is also suitable for differential diagnosis or for identifying HHV6-induced cardiomyopathy.

It is thus possible, depending on the disease to be diagnosed, to employ different microRNAs in diagnostic profiles of different microRNAs. However, it is also possible to employ a more complex diagnostic profile of different microRNAs for the diagnosis of the different diseases to be diagnosed. This has the effect that, using a single diagnostic profile, it is possible to make the most diverse diagnoses, i.e. such a profile can be used universally within the given task.

Various groups of non-ischemic cardiomyopathies or storage diseases of the heart were established, the differentiation of which from one another can be achieved using the microRNAs listed above: adenovirus-induced cardiomyopathy, enterovirus-(coxsackievirus-)induced cardiomyopathy, HHV6-virus-induced cardiomyopathy, chromosomally integrated HHV6 (ciHHV6)-induced cardiomyopathy, erythrovirus-induced cardiomyopathy, myocarditis with cardiac giant cells and cardiac amyloidosis. As illustrated, differentiation of lambda and ATTR amyloidosis may optionally be carried out.

In an embodiment, only diseases of man are considered. Accordingly, the microRNAs of the above list are, in principle, to be understood as human sequences.

In one variant, the microRNAs to be employed as marker in the context of a diagnostic profile are selected from the group consisting of all those microRNAs, those that have been assigned a score of 1 and/or 2 and/or a significance value of 1 and/or 2 for the differentiation between patients requiring, for the diagnostic identification of a myocardial disorder, myocardial biopsy, and patients not requiring myocardial biopsy, or for the diagnosis of a specific non-ischemic cardiomyopathy or a specific storage disease of the heart in the examinations illustrated in the examples given below.

In one variant, the nucleic acids to be employed as marker are selected from the group not comprising microRNAs that have been assigned a score of 3 oder 4 and/or a significance value of 4 or 5 for the differentiation between patients requiring, for the diagnostic identification of a myocardial disorder, myocardial biopsy, and patients not requiring myocardial biopsy, or for the diagnosis of a specific non-ischemic cardiomyopathy or a specific storage disease of the heart in the examinations illustrated in the examples given below. Here, individual, a plurality or all of these mRNAs that have been assigned scores of 3 or 4 and/or significance values of 4 or 5 can be excluded from the scope of protection.

In one variant, the in-vitro method is performed by carrying out the steps illustrated below. Initially, a sample, optionally pre-processed, from a patient suspected of suffering from non-ischemic cardiomyopathy or a storage disease of the heart is provided. Directly or after prior biochemical modification, the sample is then brought into contact with at least two probes, the probes each comprising a sequence which corresponds to the sequences of the microRNAs of the lists above or is complementary thereto. This contact is carried out under conditions allowing hybridization between, on the one hand, microRNAs present in the sample and, on the other hand, the probes. It is then determined if there has been hybridization between the microRNAs of the sample and the probes. Using this hybridization result, it is then determined which microRNA(s) is/are present in the sample.

The sample may be a blood serum/blood plasma sample of a patient. Alternatively, the sample may be a total RNA extract originating from a blood serum/blood plasma sample of the patient which for its part consists partially of microRNAs. Here, it is immaterial whether the RNA is isolated directly from the whole blood serum/blood plasma sample or whether specific extraction of the exosomes, which only contain a defined proportion of the circulating microRNA, is carried out beforehand. Before the method is carried out, this sample can be used to obtain an RNA extract for use as sample in the presently claimed method. It is also possible to detect the circulating microRNA directly in the blood serum/blood plasma samples.

In an embodiment, confirmation of hybridization is carried out in a semiquantitative or quantitative manner.

Use of the microRNAs mentioned above for microRNA profile diagnostics allows early identification of patients suffering from non-ischemic cardiomyopathy, a storage disease of the heart or an infection of the myocardium, even without myocardial biopsy, and subsequent classification into a certain subgroup. Following myocardial biopsy diagnosis, the respective pathological state can then be treated correspondingly in a targeted manner.

To this end, in an embodiment, the expression rates of the microRNAs mentioned above are determined in a quantitative or semiquantitative manner. Elevated expression of certain microRNAs and/or reduced expression of other certain microRNAs may then be used to diagnose a certain non-ischemic cardiomyopathy or a storage disease of the heart.

Here, the nucleic acids mentioned above (microRNAs) have been found to be particularly suitable for specific diagnostics of individual non-ischemic cardiomyopathies and storage diseases of the heart. Some of the nucleic acids are so specific for certain disorders to be diagnosed that they on their own can be used as a specific marker. Other nucleic acids are not specific for an individual disorder to be diagnosed but, for example, for two disorders to be diagnosed. Accordingly, such nucleic acids are, in an embodiment, not employed on their own but in combination with other nucleic acids from the list above. But even in the case of specific nucleic acids, in order to obtain a more accurate diagnosis, it is recommended not to employ them on their own but in combination with other nucleic acids from the above list. In this manner, it is possible to improve the validity of diagnostic tests.

Instead of the microRNAs mentioned, it is also possible to use nucleic acid molecules comprising a sequence identical to or complementary with the microRNAs mentioned above.

Aspects of the invention also relate to the use of the microRNAs listed above in the context of diagnostic profiles for the analysis of a blood serum/blood plasma sample of a patient to be able to differentiate between patients requiring, for the diagnostic identification of a myocardial disorder, myocardial biopsy, and patients not requiring myocardial biopsy, and for the optional differential diagnostic differentiation between individual non-ischemic cardiomyopathies or storage diseases of the heart.

Aspects of the invention also relate to a diagnostic system for differentiating between patients requiring, for the diagnostic identification of a myocardial disorder, myocardial biopsy, and patients not requiring myocardial biopsy, and for the optional differential diagnostic differentiation between individual non-ischemic cardiomyopathies or storage diseases of the heart. Such a diagnostic system comprises at least two probes each comprising a sequence which corresponds to a sequence of a microRNA from the group consisting of the microRNAs below, or is complementary thereto: hsa-miR-487b-3p (SEQ ID NO: 23), hsa-miR-494-3p (SEQ ID NO: 24), hsa-miR-889-3p (SEQ ID NO: 29), mmu-miR-495-3p (SEQ ID NO: 30). With regard to the term “corresponds”, reference is made to the above comments on the term “identical”, which are to be used in an analogous manner.

In one variant, the diagnostic system additionally comprises at least three probes each comprising a sequence which corresponds to a sequence of a microRNA from the group consisting of the microRNAs below, or is complementary thereto: hsa-miR-1274B (SEQ ID NO: 31), hsa-miR-4449 (SEQ ID NO: 37), hsa-miR-4674 (SEQ ID NO: 38), hsa-miR-5096 (SEQ ID NO: 39), hsa-miR-548c-5p (SEQ ID NO: 41), hsa-miR-1180-3p (SEQ ID NO: 43), hsa-miR-1-3p (SEQ ID NO: 44), hsa-miR-16-5p (SEQ ID NO: 47), hsa-miR-3138 (SEQ ID NO: 52), hsa-miR-3622b-5p (SEQ ID NO: 53), hsa-miR-3922-5p (SEQ ID NO: 54), hsa-miR-4274 (SEQ ID NO: 56), hsa-miR-4303 (SEQ ID NO: 57), hsa-miR-4419b (SEQ ID NO: 58), hsa-miR-4429 (SEQ ID NO: 59), hsa-miR-4467 (SEQ ID NO: 60), hsa-miR-4651 (SEQ ID NO: 61), hsa-miR-4′716-5p (SEQ ID NO: 62), hsa-miR-548b-5p (SEQ ID NO: 63), hsa-miR-597-5p (SEQ ID NO: 65), hsa-miR-923 (SEQ ID NO: 70), mmu-miR-374-5p (SEQ ID NO: 71), miR-1274A (SEQ ID NO: 76), hsa-miR-1274B (SEQ ID NO: 77), hsa-miR-223-5p (SEQ ID NO: 88), hsa-miR-328-3p (SEQ ID NO: 95), hsa-miR-378-5p (SEQ ID NO: 97), hsa-miR-423 (SEQ ID NO: 98), hsa-miR-4286 (SEQ ID NO: 99), hsa-miR-4535 (SEQ ID NO: 101), hsa-miR-210-3p (SEQ ID NO: 112), hsa-miR-103b (SEQ ID NO: 117), hsa-miR-1274B (SEQ ID NO: 120), hsa-miR-138-1-3p (SEQ ID NO: 121), hsa-miR-146b-3p (SEQ ID NO: 122), hsa-miR-190a-5p (SEQ ID NO: 123), hsa-miR-26b-3p (SEQ ID NO: 125), hsa-miR-3617-5p (SEQ ID NO: 127), hsa-miR-3663-3p (SEQ ID NO: 129), hsa-miR-3922-5p (SEQ ID NO: 131), hsa-miR-424-5p (SEQ ID NO: 132), hsa-miR-4429 (SEQ ID NO: 134), hsa-miR-4698 (SEQ ID NO: 135), hsa-miR-4726-3p (SEQ ID NO: 136), hsa-miR-4743-5p (SEQ ID NO: 137), hsa-miR-597-5p (SEQ ID NO: 142), hsa-miR-629-5p (SEQ ID NO: 145), hsa-miR-758-3p (SEQ ID NO: 146), mmu-miR-374-5p (SEQ ID NO: 147), hsa-miR-146b-3p (SEQ ID NO: 151), hsa-miR-3156-5p (SEQ ID NO: 153), hsa-miR-4535 (SEQ ID NO: 157), hsa-miR-487b-3p (SEQ ID NO: 160), hsa-miR-494-3p (SEQ ID NO: 161), hsa-miR-511-5p (SEQ ID NO: 162), hsa-miR-548c-5p (SEQ ID NO: 165), hsa-miR-889-3p (SEQ ID NO: 169), mmu-miR-374-5p (SEQ ID NO: 175).

In an embodiment, the diagnostic system comprises at least or exactly 2, 3, 4, 5, 6, 7, 8, 9, 10, 11, 12, 13, 14, 15, 20, 25, 30, 35, 40, 45, 50 of the microRNAs listed above as probes, each of which may be present in the diagnostic system in a plurality of copies. Furthermore, it is possible for all microRNAs of the above list in identical or complementary sequence to be present in the diagnostic system as probe. Thus, the diagnostic system has a quantifiable number of diagnostically relevant microRNA sequences which, in the system, represent a diagnostically relevant microRNA profile.

In an embodiment, the diagnostic system is present in the form of a kit for carrying out a polymerase chain reaction (PCR), where the probes are provided as primers in solution. In this manner, it is particularly easy to work quantitatively, since the kit can be used to carry out a quantitative PCR. By using carrier plates having a large number of wells (for example 384 wells), such a system allows numerous PCRs to be performed at the same time, making this system suitable even for a large number of probes.

In one variant, the expression levels are determined by measuring the individual target microRNAs in comparision to one or more constitutively expressed microRNAs, so-called housekeeping microRNAs, in the respective blood serum/blood plasma sample.

In an alternative preferred embodiment, the diagnostic system is present in the form of a nucleic acid chip. Here, in particular, at least 5 probes for each nucleic acid to be detected may be provided on the chip. In this manner, the expression of microRNAs may preferably be measured semiquantitatively. Using a chip has the effect that is is possible to detect simultaneously the presence of a large number of different microRNAs in the sample examined. Here, the number of simultaneously detectable microRNAs is far greater than in the case of a PCR. In particular in cases with a relatively large number of probes (for example more than 25 probes), the use of a chip is particularly appropriate for reasons of efficiency.

Aspects of the invention also relate to a medicament for the treatment of non-ischemic cardiomyopathies or storage diseases of the heart, comprising, as pharmaceutically active substance, at least one nucleic acid comprising a sequence identical to or complementary with the sequence of one of the microRNAs listed in connection with the diagnostic system.

Such a medicament may serve to increase the content of a certain microRNA in the blood and/or in the cells of a patient to enhance the positive properties of this microRNA with respect to a specific pathological state. It may also serve to eliminate a certain microRNA whose content is elevated in a certain pathological state by hybridization or a comparable interaction to counteract the negative effect of this microRNA on the pathological state in question.

Preferably, the selected nucleic acid or group of nucleic acids is the only pharmaceutically active component of the medicament in question.

Preferably, the medicament is suitable not only for therapeutic purposes but also for diagnostic purposes.

Preferred embodiments of the method shown above and illustrated below in connection with the examples, where microRNAs are employed, can also be applied analogously to the medicament claimed. This relates in particular to the selection of appropriate nucleic acid sequences or subgroups of sequences using microRNAs mentioned as being preferably employed.

Aspects of the invention also relate to the therapeutic utilization of the microRNAs shown or nucleic acid sequences identical with or complementary to these microRNAs, in particular for the treatment or therapy of non-ischemic cardiomyopathies or storage diseases of the heart.

Preferred embodiments of the use of the microRNAs shown above and illustrated below in connection with the examples can also be applied analogously to a corresponding therapeutic application.

Aspects of the invention also relate to a diagnostic method, carried out in vivo, which can be performed analogously to the in-vitro method described above, and which does not require a sample to be taken from a patient.

### BRIEF DESCRIPTION OF THE DRAWINGS

Using FIGS. and examples, aspects of the present invention are illustrated in more detail.

FIG. 1A is a representation of the expression level of a first microRNA marker depending on the pathological state in question,

FIG. 1B is a representation of the expression level of a second microRNA marker depending on the pathological state in question,

FIG. 1C is a representation of the expression level of a third microRNA marker depending on the pathological state in question,

FIG. 2A is a first schematic representation of the diagnostic application of profiles of circulating microRNAs for the identification and differential diagnosis of cardiomyopathy patients requiring biopsy and

FIG. 2B is a second schematic representation of the diagnostic application of profiles of circulating microRNAs for the identification and differential diagnosis of cardiomyopathy patients requiring biopsy.

### DETAILED DESCRIPTION

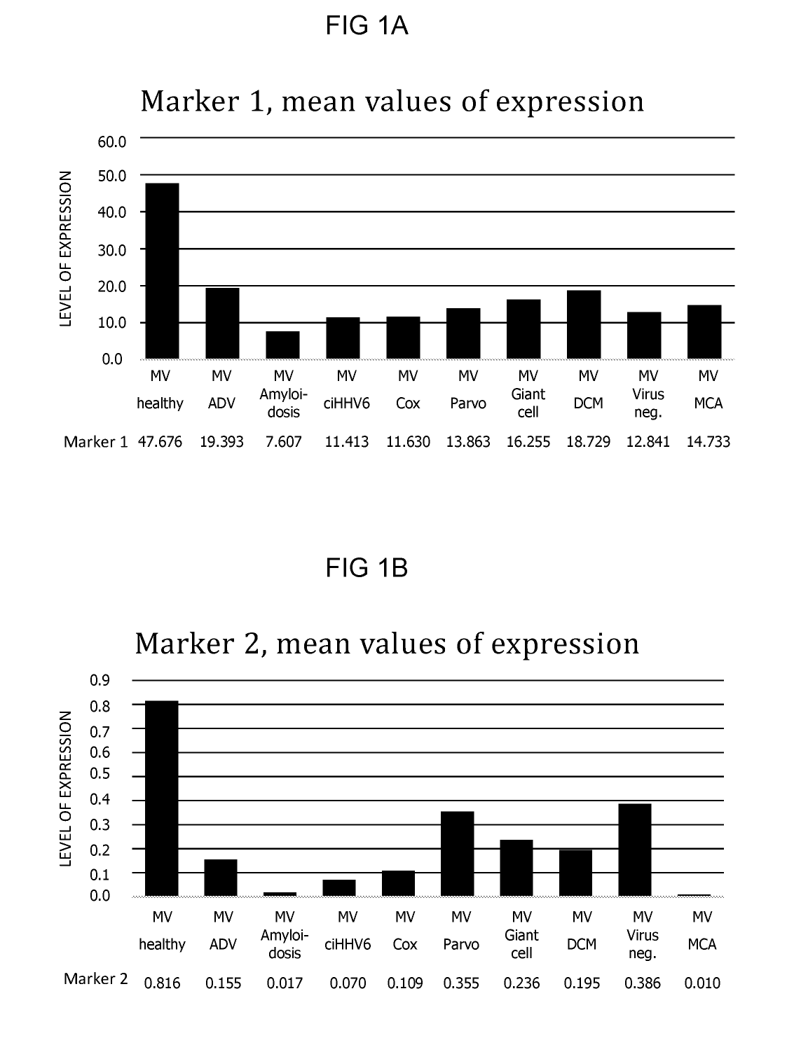

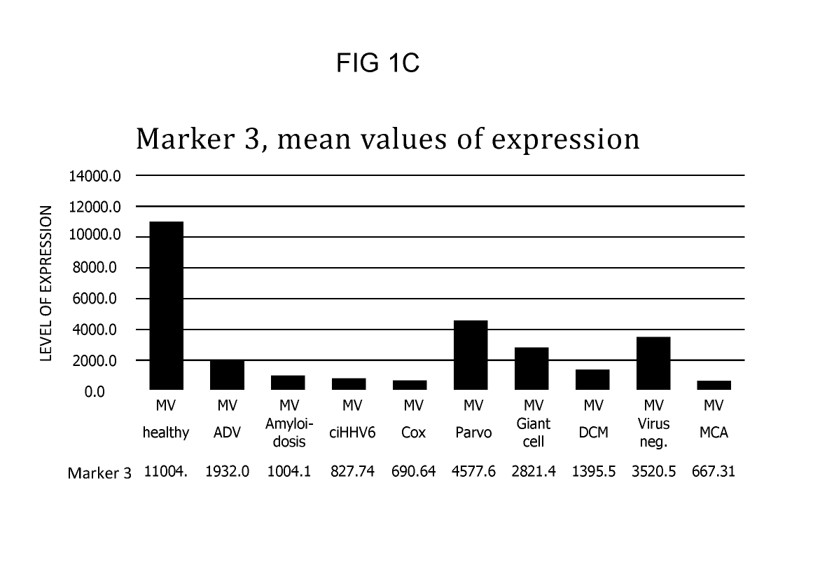

FIG. 1A shows a representation of the mean expression values of a first microRNA marker for a healthy control group and for patients with different pathological states. Here, the first marker used was the microRNA hsa-mir-197-3p (SEQ ID NO: 12). In the case of healthy patients, this microRNA has a mean expression level of almost 50.0 auf. For patients suffering from adenovirus-induced cardiomyopathy (ADV), a mean (MW) of almost 20 was identified. For patients suffering from amyloidosis of the heart (Amyloidosis), for patients suffering from ciHHV6-induced cardiomyopathy (ciHHV6), for patients suffering from coxsackievirus-induced cardiomyopathy (Cox), for patients suffering from parvovirus-induced cardiomyopathy (Parvo), for patients suffering from giant cell cardiomyopathy (Giant cells), for patients suffering from dilated cardiomyopathy (DCM), for patients suffering from cardiomyopathy without viral inflammation of the heart (Virus neg) and for patients suffering from acute myocarditis (MCA), the expression level was in each case lower than in the case of patients suffering from adenovirus-induced cardiomyopathy. Overall, FIG. 1A shows that the microRNA used as marker 1 is a good marker for differentiation between healthy and ill patients. Accordingly, this microRNA is highly suitable for use in the context of a diagnostic profile as marker for identifying patients not requiring myocardial biopsy.

FIG. 1B shows a representation of the mean expression values of a second microRNA marker, namely the microRNA hsa-let-7f-5p (SEQ ID NO: 10). For this microRNA, too, the mean of the expression level for healthy patients differs significantly from the expression level of patients suffering from cardiomyopathy. With regard to the abbreviations used in FIG. 1B, reference is made to the comments on FIG. 1A. Accordingly, the microRNA used as marker 2 is likewise a good marker for identifying patients not requiring myocardial biopsy.

FIG. 1C shows the mean expression values of a third marker, namely the microRNA hsa-mir-223-3p (SEQ ID NO: 14). For this microRNA, too, the expression level for healthy patients is significantly higher than for patients suffering from a specific cardiomyopathy. With regard to the abbreviations used, reference is once more made to the comments on FIG. 1A. Accordingly, marker 3 is likewise a suitable microRNA marker for differentiating between patients not requiring myocardial biopsy and patients requiring myocardial biopsy for further diagnostic examination.

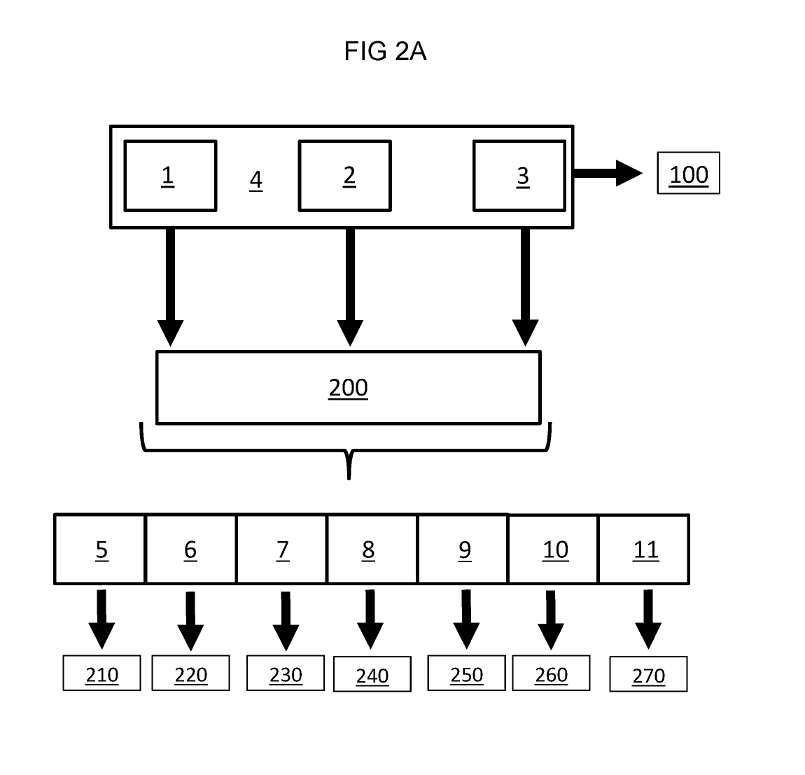

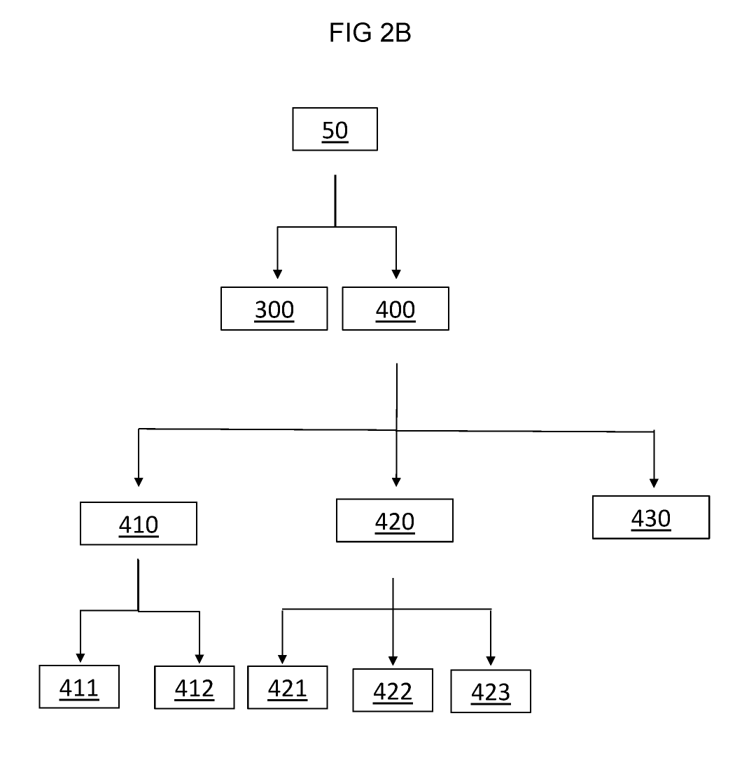

FIG. 2A shows a schematic representation of an exemplary diagnostic application of diagnostic profiles of microRNAs circulating in blood serum/blood plasma for the identification and differential diagnosis of cardiomyopathy patients requiring biopsy. First, a blood serum/blood plasma sample of a patient is tested for presence of a first marker 1, a second marker 2 and a third marker 3. These three markers may, for example, be the markers whose expression levels are shown in FIGS. 1A to 1C.

The first marker 1, the second marker 2 and the third marker 3 have been summarized as diagnostic profile 4. If a high expression level of the first marker 1, the second marker 2 and the third marker 3 is found, this shows that the patient is healthy. In this case a first diagnosis 100 is made according to which the patient is healthy and does not require myocardial biopsy.

If, however, the expression level of one of the three markers 1, 2, 3 is not on a high level (compare FIGS. 1A to 1C), a second diagnosis 200 is made according to which a myocardial disorder is presumably present. A more accurate diagnostic identification of the myocardial disorder ultimately requires a myocardial biopsy. However, to make this myocardial biopsy easier, preclassification of the patient examined into a patient group is carried out. To this end, the sample taken from the patient is analyzed in more detail using a second diagnostic profile 5, a third diagnostic profile 6, a fourth diagnostic profile 7, a fifth diagnostic profile 8, a sixth diagnostic profile 9, a seventh diagnostic profile 10 and an eighth diagnostic profile 11.

Using the second diagnostic profile 5, it can be determined if the patient is suffering from adenovirus-induced cardiomyopathy. If this is the case, a corresponding third diagnosis 210 is made. During the myocardial biopsy that follows, one can then look specifically for presence of adenoviruses, which simplifies the corresponding diagnosis and makes the diagnosis result available more quickly.

Using the third diagnostic profile 6, it is possible to determine if the patient is suffering from coxsackievirus-induced cardiomyopathy. If this is the case, a corresponding fourth diagnosis 220 is made.

Using the fourth diagnostic profile 7, it is possible to determine if the patient is suffering from parvovirus-induced cardiomyopathy. If this is the case, a corresponding fifth diagnosis 230 is made.

Using the fifth diagnostic profile 8, it is possible to determine if the patient is suffering from (ci)HHV6 virus-induced cardiomyopathy. If this is the case, a corresponding sixth diagnosis 240 is made.

Using the sixth diagnostic profile 9, it is possible to determine if the patient is suffering from inflammatory dilated cardiomyopathy. If this is the case, a corresponding seventh diagnosis 250 is made.

Using the seventh diagnostic profile 10, it is possible to determine if the patient is suffering from cardiac sarcoidosis or giant cell myocarditis. If this is the case, a corresponding eighth diagnosis 260 is made.

Using the eighth diagnostic profile 11, it is possible to determine if the patient is suffering from amyloidosis of the heart. If this is the case, a corresponding ninth diagnosis 270 is made.

Each of the diagnostic profiles 4, 5, 6, 7, 8, 9, 10, 11 comprises at least three microRNAs as marker. However, it is possible for the individual diagnostic profiles to comprise even more microRNAs.

The sample to be examined can be analyzed simultaneously using all diagnostic profiles 4, 5, 6, 7, 8, 9, 10, 11. Sequential evaluation may then be carried out to establish whether the patient is healthy or ill and, if he/she is ill, which form of cardiomyopathy he/she is suffering from.

Alternatively, it would be also possible to use initially just one diagnostic profile for the analysis of the patient sample and, depending on the analysis result, to perform subsequent further analysis steps with further diagnostic profiles. However, with regard to workflow and sample handling, this is frequently more complicated than simultaneous analysis of all diagnostic profiles.

FIG. 2B shows a second schematic representation of a possible diagnostic application of diagnostic profiles of microRNAs circulating in blood serum/blood plasma for identification and differential diagnosis of cardiomyopathy patients requiring biopsy.

First, a sample 50 of a patient is provided, it being unknown if the patient is healthy or suffering from cardiomyopathy. Accordingly, in a first diagnostic step, either a first diagnosis 300 “patient healthy” or a second diagnosis 400 “patient ill” is made.

If the patient is healthy, sample 50 of the patient does not require further examination. If, however, the patient is ill, sample 50 of the patient is analyzed in more detail to establish the type of cardiomyopathy the patient is suffering from. During this second diagnostic step, for example, a third diagnosis 410 may then be made according to which the patient is suffering from amyloidosis of the heart. Alternatively, in the second diagnostic step a fourth diagnosis 420 may also be made according to which the patient is suffering from virus-induced cardiomyopathy.

Finally, in this second diagnostic step a fifth diagnosis 430 may also be made according to which the patient is suffering from inflammatory dilated cardiomyopathy.

Then, in a further diagnostic step, a subclassification of the pathological states determined beforehand may be made. If, for example, it was found in the third diagnosis 410 that the patient suffers from amyloidosis of the heart, it may be established in a third diagnostic step which type of amyloidosis this is. During the third diagnostic step, a sixth diagnosis 411 may be made according to which the patient suffers from ATTR amyloidosis. Alternatively, during the third diagnostic step a seventh diagnosis 412 may also be made according to which the patient suffers from lambda amyloidosis.

If, in the second diagnostic step, the fourth diagnosis 420 has shown that the patient suffers from virus-induced cardiomyopathy, in the third diagnostic step it may be established by an eighth diagnosis 421 that the patient suffers from enterovirus-induced cardiomyopathy. Alternatively, a ninth diagnosis 422 may establish that the patient suffers from (ci)HHV6-induced cardiomyopathy. Finally, in the third diagnostic step, it may be established by a tenth diagnosis 423 that the patient suffers from coxsackievirus-induced cardiomyopathy.

The first diagnostic step, the second diagnostic step and the third diagnostic step can—as already mentioned in connection with the representation of FIG. 2 A—be carried out simultaneously by using different diagnostic profiles.

### WORKING EXAMPLES

To be able to state, in the examples illustrated below, whether the expression of the microRNAs studied is upregulated or downregulated, the microRNAs in which more than 95% of all blood serum/blood plasma samples were expressed relatively constantly were identified. To this end, the procedure described in connection with Example 1 was adopted. Here, both cardiologically healthy blood donors (Example 1) and patients suffering from cardiomyopathies, diagnosable by myocardial biopsies, of viral inflammatory etiology (Examples 2 to 8) were considered. These constitutively expressed microRNAs are also referred to as housekeeping microRNAs. These housekeeping microRNAs were then used as normalization sequences for relative statements on the expression modification of the other microRNAs examined.

The housekeeping microRNAs identified are listed in Table 1 below. The expression of disease-relevant microRNAs according to Examples 2 to 8 was normalized against one or more housekeeping microRNAs to determine the diagnosis-specific expression level (compare, for example, FIG. 1).

TABLE 1
Constitutively expressed microRNAs (housekeeping microRNAs)
as normalization sequences for measuring differentially
regulated microRNAs in blood serum/blood plasma samples

| SEQ ID | | Regulation |
|---|---|---|
| NO | microRNA | ↑↓ |
| 1 | hsa-miR-10a-5p | HK |
| 2 | hsa-miR-10b-5p | HK |
| 3 | hsa-miR-130b-3p | HK |
| 4 | hsa-miR-148b-3p | HK |
| 5 | hsa-miR-152-3p | HK |
| 6 | hsa-miR-185-5p | HK |
| 7 | hsa-miR-27b-3p | HK |
| 8 | hsa-miR-30a-3p | HK |
| 9 | hsa-miR-335-5p | HK |

HK = Housekeeping

### Example 1

### Diagnosis of Patients Not Suspected of Suffering from Viral Inflammatory Myocardial Disorder or Amyloidosis Using a Blood Serum/Blood Plasma Sample (Identification of Patients Not Requiring Biopsy)

Blood serum/blood plasma samples were taken from several human patients not suffering from any of the cardiomyopathies of viral inflammatory etiology presently diagnosable by myocardial biopsy or not yet displaying any diagnosable symptoms (control group).

Using standard methods generally known to the person skilled in the art, total RNA extracts (i.e. extracts of the total RNA of the respective blood serum/blood plasma sample) also containing microRNA components were obtained from the individual samples. These total RNA extracts were then examined by two variants for the presence of certain microRNAs.

Variant 1 (RNA Chip/Microparticle)

The RNA of the total RNA extract was labeled with biotin, applied to an RNA chip and incubated for 16 hours. Such an RNA chip is sometimes also referred to as DNA chip since it contains DNA probes. However, these are intended for RNA detection, therefore in the present case the term “RNA chip” is used. During incubation hybridization of the microRNA present in the total RNA extract with complementary DNA probes took place on the RNA chip. Hybridized microRNA molecules were then detected using the fluorescent dye streptavidin-phycoerythrin, which binds to biotin. By determining the fluorescence intensity of the bound streptavidin-phycoerythrin, it was possible to determine the amount of hybridized microRNA in a semiquantitative manner.

Analogously, the hybridization of the microRNAs of interest may also be performed on differently-colored microparticles each carrying one or more complementary DNA or RNA probes on their surface. Subsequent to the binding, washing and labeling steps, detection may then be carried out using microscopic or flow cytometry methods.

For the individual steps of the method, standard buffers recommended by the RNA chip manufacturer or specially prepared solution were used. These are generally known to the person skilled in the art. “Quantigene” kits from Affymetrix were used for direct microRNA detection, and microparticles and kits from Luminex were used for detection by flow cytometry.

Variant 2 (PCR Gene Card)

The microRNA present in the total RNA extract was used to synthesize cDNA. Depending on the amount of cDNA obtained, the cDNA was preamplified, if required. The cDNA was then applied to a card and quantitative PCR was carried out. In this manner, relative quantification of the individual cDNAs could be performed in real time via the relative fluorescence compared with constitutively expressed microRNAs.

For the individual steps of the method, standard buffers recommended by the respective manufacturer of the gene cards were used. These are generally known to the person skilled in the art. Among others, gene cards from Applied Biosystems were used.

By applying the methods according to variant 1 and variant 2 in a complementary fashion, numerous microRNAs were identified whose expression was modified in comparison to samples from patients suffering from non-ischemic cardiomyopathies or a storage disease of the heart.

Table 2 lists the microRNAs identified whose expression was modified in the control group in comparison to examined samples from cardiomyopathy patients. Furthermore, this table states the type of modification. To this end, taking into account the results illustrated in the examples below, a score was calculated and it was determined whether expression of the microRNA in question was up- or downregulated. The exact meaning of the score is discussed in connection with Example 2.

Furthermore, a significance value was calculated which states the significance of the microRNA in question for the question studied. The significance value reflects the type of expression modification, the detectability of the microRNA in question on various detection platforms, the score and other parameters. The significance value can have values from 1 to 6, 1 meaning high significance and 6 meaning low significance.

The identified microRNAs of Table 2 are particularly suitable for identifying patients who, at the time of the examination, do not require examination by biopsy, or where the decision of whether a biopsy should be performed may be taken at a later examination time, should the clinical parameters or the pattern of circulating microRNAs change.

The identified microRNAs of Table 2 are also particularly suitable for sequential differential diagnosis by the procedure described in an exemplary manner in FIGS. 2A and 2B in order to identify patients requiring biopsy and then subjecting them to an optional subclass analysis.

TABLE 2
Expression modifications of microRNAs for the diagnosis
of patients currently not requiring biopsy for the pathological
state of viral inflammatory cardiomyopathies.

| SEQ ID | | Regulation | Significance | |
|---|---|---|---|---|
| NO | microRNA | ↑↓ | value | Score |
| 10 | hsa-let-7f-5p | up | 1 | 1 |
| 11 | hsa-miR-103a-3p | up | 4 | 2 |
| 12 | hsa-miR-197-3p | up | 1 | 1 |
| 13 | hsa-miR-215-5p | up | 2 | 4 |
| 14 | hsa-miR-223-3p | up | 1 | 1 |
| 15 | hsa-miR-23a-3p | up | 1 | 1 |
| 16 | hsa-miR-330-3p | up | 3 | 1 |
| 17 | hsa-miR-337-5p | up | 3 | 1 |
| 18 | hsa-miR-339-3p | up | 3 | 1 |
| 19 | hsa-miR-339-5p | up | 3 | 1 |
| 20 | hsa-miR-411-5p | down | 4 | 3 |
| 21 | hsa-miR-454-3p | up | 3 | 1 |
| 22 | hsa-miR-485-3p | up | 5 | 3 |
| 23 | hsa-miR-487b-3p | up | 5 | 3 |
| 24 | hsa-miR-494-3p | up | 5 | 3 |
| 25 | hsa-miR-505-3p | up | 3 | 1 |
| 26 | hsa-miR-628-5p | up | 5 | 3 |
| 27 | hsa-miR-671-3p | up | 5 | 3 |
| 28 | hsa-miR-744-5p | up | 3 | 1 |
| 29 | hsa-miR-889-3p | up | 5 | 3 |
| 30 | mmu-miR-495-3p | up | 3 | 1 |

In one variant, the invention relates to the use of the microRNAs having a score of 1 and/or 2 and/or a significance value of 1 and/or 2 in Table 2 individually or in any combination with one another as marker for identifying patients not requiring biopsy. Here, a combination of a score of 1 with a significance value of 1 indicates particularly suitable microRNAs.

### Example 2

### Diagnosis of Adenovirus-Induced Cardiomyopathies Using a Using a Blood Serum/Blood Plasma Sample

The procedure of Example 1 was adopted, except that the blood serum/blood plasma samples were taken from patients suffering from adenovirus-induced cardiomyopathy.

Table 3 below lists the microRNAs identified whose expression is modified in adenovirus-induced cardiomyopathy. Furthermore, this table states the type of this modification. To this end, taking into account the results illustrated in the examples, another score was calculated and it was determined whether expression of the microRNA in question was up- or downregulated.

A score of 1 means that the expression modification observed is specific for one of the non-ischemic cardiomyopathies or storage diseases of the heart studied and is markedly changed only in the case of this disease. Accordingly, microRNAs having a score of 1 are specific markers for adenovirus-induced cardiomyopathy. These microRNAs can exert their marker function by increased expression (upregulation) or reduced expression (downregulation). In other words, a score of 1 denotes microRNAs allowing unambiguous identification of a pathological state, even if the type of the observed modification of expression is unknown.

A score of 2 means that the expression modification observed is specific for two of the non-ischemic cardiomyopathies or storage diseases of the heart studied and is markedly changed only in the case of these two diseases. The expression here is increased in the case of the first of these two diseases and reduced in the case of the second of these two diseases. Accordingly, microRNAs having a score of 2 are specific markers for adenovirus-induced cardiomyopathy if it is additionally known whether their expression is upregulated or else downregulated. In other words, a score of 2 denotes microRNAs allowing unambiguous identification of a pathological state provided that, in addition, the type of observed modification of expression is known.

A score of 3 means that the expression modification observed is significant for a plurality of the non-ischemic cardiomyopathies or storage diseases of the heart studied. Here, in the case of two diseases the expression is modified in the same sense, i.e. either elevated or reduced. In all other diseases where the expression modification is likewise significant, the expression is modified in the opposite sense, i.e. either reduced or elevated. Therefore, on their own, microRNAs having a score of 3 are unspecific markers for adenovirus-induced cardiomyopathy since they might also indicate a different pathological state. However, together with a further microRNA marker, either specific or else likewise unspecific, an unambiguous determination of adenovirus-induced cardiomyopathy is already possible. However, in the case of a further unspecific marker this only applies if its expression is modified in other further diseases than those further diseases where the expression of the first unspecific marker is modified.

A score of 4 means that the expression modification observed is significant for exactly two of the non-ischemic cardiomyopathies or storage diseases of the heart studied. Here, in both cases, the expression is modified in the same sense, i.e. either elevated or reduced. Therefore, on their own, microRNAs having a score of 4 are unspecific markers for adenovirus-induced cardiomyopathy since they might also indicate a different pathological state. However, together with a further microRNA marker, either specific or else likewise unspecific, an unambiguous determination of adenovirus-induced cardiomyopathy is already possible. However, in the case of a further unspecific marker this only applies if its expression is modified in another further disease than the further disease where the expression of the first unspecific marker is modified in the same sense.

With regard to the significance values, which were also determined, reference is made to the comments above.

TABLE 3
Expression modifications of microRNAs
in adenovirus-induced cardiomyopathy.

| SEQ ID | | Regulation | Significance | |
|---|---|---|---|---|
| NO | microRNA | ↑↓ | value | Score |
| 31 | hsa-miR-1274B | down | 6 | 4 |
| 32 | hsa-miR-136-3p | up | 3 | 1 |
| 33 | hsa-miR-187-3p | down | 4 | 1 |
| 34 | hsa-miR-199a-5p | up | 5 | 3 |
| 35 | hsa-miR-215-5p | down | 2 | 4 |
| 36 | hsa-miR-422a | down | 4 | 4 |
| 37 | hsa-miR-4449 | up | 3 | 1 |
| 38 | hsa-miR-4674 | up | 3 | 1 |
| 39 | hsa-miR-5096 | up | 3 | 1 |
| 40 | hsa-miR-548c-3p | up | 5 | 3 |
| 41 | hsa-miR-548c-5p | up | 6 | 3 |
| 42 | hsa-miR-650 | up | 1 | 1 |

In one variant, the invention relates to the use of the microRNAs having a score of 1 and/or 2 and/or a significance value of 1 and/or 2 in Table 3 individually or in any combination with one another as marker for identifying adenovirus-induced cardiomyopathy. Here, a combination of a score of 1 with a significance value of 1 indicates particularly suitable microRNAs.

### Example 3

### Diagnosis of Enterovirus-Induced (Coxsackievirus-Induced) Cardiomyopathies Using a Blood Serum/Blood Plasma Sample

The procedure of Example 1 was adopted, except that the blood serum/blood plasma samples were taken from patients suffering from enterovirus-(coxsackievirus-)induced cardiomyopathy. The microRNAs identified, whose expression is modified in such an enterovirus-(coxsackievirus-) induced cardiomyopathy, are listed in Table 4 below. With regard to the scores and significance values, reference is made to the comments on Examples 1 and 2.

TABLE 4
Expression modifications of microRNAs in enterovirus-
(coxsackievirus-)induced cardiomyopathy.

| SEQ ID | | Regulation | Significance | |
|---|---|---|---|---|
| NO | microRNA | ↑↓ | value | Score |
| 43 | hsa-miR-1180-3p | down | 3 | 1 |
| 44 | hsa-miR-1-3p | up | 3 | 1 |
| 45 | hsa-miR-140-5p | up | 3 | 1 |
| 46 | hsa-miR-150-3p | up | 3 | 1 |
| 47 | hsa-miR-16-5p | down | 4 | 1 |
| 48 | hsa-miR-215-5p | down | 2 | 4 |
| 49 | hsa-miR-25-3p | down | 1 | 1 |
| 50 | hsa-miR-30a-5p | down | 5 | 3 |
| 51 | hsa-miR-30e-5p | up | 3 | 1 |
| 52 | hsa-miR-3138 | up | 3 | 1 |
| 53 | hsa-miR-3622b-5p | up | 3 | 1 |
| 54 | hsa-miR-3922-5p | up | 5 | 2 |
| 55 | hsa-miR-422a | up | 4 | 4 |
| 56 | hsa-miR-4274 | down | 3 | 1 |
| 57 | hsa-miR-4303 | down | 3 | 1 |
| 58 | hsa-miR-4419b | down | 3 | 1 |
| 59 | hsa-miR-4429 | up | 5 | 3 |
| 60 | hsa-miR-4467 | down | 3 | 1 |
| 61 | hsa-miR-4651 | up | 3 | 1 |
| 62 | hsa-miR-4716-5p | up | 3 | 1 |
| 63 | hsa-miR-548b-5p | down | 3 | 1 |
| 64 | hsa-miR-548c-3p | up | 5 | 3 |
| 65 | hsa-miR-597-5p | up | 5 | 3 |
| 66 | hsa-miR-601 | up | 4 | 3 |
| 67 | hsa-miR-629-3p | down | 3 | 1 |
| 68 | hsa-miR-660-5p | down | 6 | 3 |
| 69 | hsa-miR-886-3p | up | 5 | 3 |
| 70 | hsa-miR-923 | up | 3 | 1 |
| 71 | mmu-miR-374-5p | up | 6 | 3 |

In one variant, the invention relates to the use of the microRNAs having a score of 1 and/or 2 and/or a significance value of 1 and/or 2 in Table 4 individually or in any combination with one another as marker for identifying an enterovirus-(coxsackievirus-)induced cardiomyopathy. Here, a combination of a score of 1 with a significance value of 1 indicates particularly suitable microRNAs.

### Example 4

### Diagnosis of Amyloidosis of the Heart Using a Blood Serum/Blood Plasma Sample

The procedure of Example 1 was adopted, except that the blood serum/blood plasma samples were taken from patients suffering from amyloidosis of the heart.

Amyloidosis is a typical storage disease of the heart and is characterized by amyloid deposits in the myocardium.

The microRNAs identified, whose expression is modified in such an amyloidosis, are listed in Table 5 below. With regard to the scores and significance values, reference is made to the comments on Examples 1 and 2.

Some of the microRNAs in Table 5 are additionally suitable for the subclassification of two of the most frequent forms of cardiac amyloidosis (lambda amyloidosis and ATTR amyloidosis). The microRNAs suitable for subclassification are listed in Tables 6 and 7 below.

TABLE 5
Expression modifications of microRNAs
in amyloidosis of the heart.

| SEQ ID | | Regulation | Significance | |
|---|---|---|---|---|
| NO | microRNA | ↑↓ | value | Score |
| 72 | hsa-miR-103a-3p | up | 4 | 2 |
| 73 | hsa-miR-106b-5p | down | 3 | 1 |
| 74 | hsa-miR-126-3p | down | 3 | 1 |
| 75 | hsa-miR-126-5p | down | 3 | 1 |
| 76 | hsa-miR-1274A | down | 4 | 1 |
| 77 | hsa-miR-1274B | down | 6 | 4 |
| 78 | hsa-miR-132-3p | up | 3 | 1 |
| 79 | hsa-miR-140-3p | down | 4 | 1 |
| 80 | hsa-miR-142-3p | down | 3 | 1 |
| 81 | hsa-miR-146b-5p | down | 3 | 1 |
| 82 | hsa-miR-150-5p | down | 3 | 1 |
| 83 | hsa-miR-15b-5p | down | 3 | 1 |
| 84 | hsa-miR-181b-5p | down | 3 | 1 |
| 85 | hsa-miR-186-5p | down | 3 | 1 |
| 86 | hsa-miR-21-5p | down | 3 | 1 |
| 87 | hsa-miR-222-3p | down | 4 | 1 |
| 88 | hsa-miR-223-5p | down | 3 | 1 |
| 89 | hsa-miR-24-3p | down | 4 | 3 |
| 90 | hsa-miR-26b-5p | down | 3 | 1 |
| 91 | hsa-miR-30a-3p | down | 1 | 1 |
| 92 | hsa-miR-30d-5p | down | 3 | 1 |
| 93 | hsa-miR-320a | down | 3 | 1 |
| 94 | hsa-miR-320c | up | 5 | 3 |
| 95 | hsa-miR-328-3p | down | 3 | 1 |
| 96 | hsa-miR-375 | up | 3 | 1 |
| 97 | hsa-miR-378-5p | down | 3 | 1 |
| 98 | hsa-miR-423 | up | 3 | 1 |
| 99 | hsa-miR-4286 | down | 3 | 1 |
| 100 | hsa-miR-451a | down | 3 | 1 |
| 101 | hsa-miR-4535 | up | 4 | 3 |
| 102 | hsa-miR-483-5p | down | 5 | 3 |
| 103 | hsa-miR-518d-3p | up | 6 | 3 |
| 104 | hsa-miR-532-5p | down | 3 | 1 |
| 105 | hsa-miR-590-5p | down | 5 | 3 |
| 106 | hsa-miR-601 | up | 4 | 3 |
| 107 | hsa-miR-660-5p | down | 6 | 3 |
| 108 | hsa-miR-885-5p | down | 3 | 1 |
| 109 | hsa-miR-92a-3p | down | 6 | 3 |
| 110 | hsa-miR-99b-5p | down | 3 | 1 |

TABLE 6
MicroRNAs suitable for subclassification of lambda amyloidosis

| SEQ ID | | Regulation | Significance | |
|---|---|---|---|---|
| NO | microRNA | ↑↓ | value | Score |
| 73 | hsa-miR-106b-5p | down | 3 | 1 |
| 76 | hsa-miR-1274A | down | 4 | 1 |
| 91 | hsa-miR-30a-3p | down | 1 | 1 |
| 101 | hsa-miR-4535 | up | 4 | 3 |

TABLE 7
MicroRNAs suitable for subclassification of ATTR amyloidosis

| SEQ ID | | Regulation | Significance | |
|---|---|---|---|---|
| NO | microRNA | ↑↓ | value | Score |
| 79 | hsa-miR-140-3p | down | 4 | 1 |
| 84 | hsa-miR-181b-5p | down | 3 | 1 |
| 87 | hsa-miR-222-3p | down | 4 | 1 |
| 88 | hsa-miR-223-5p | down | 3 | 1 |

In one variant, the invention relates to the use of the microRNAs having a score of 1 and/or 2 and/or a significance value of 1 and/or 2 in Tables 5 to 7 individually or in any combination with one another as markers for identifying a storage disease of the heart, in particular amyloidosis of the heart. Here, a combination of a score of 1 with a significance value of 1 shows particularly suitable microRNAs.

### Example 5

### Diagnosis of Cardiac Sarcoidosis (CS)/Giant Cell Myocarditis Using a Blood Serum/Blood Plasma Sample

The procedure of Example 1 was adopted, except that the blood serum/blood plasma samples were taken from patients suffering from giant cell myocarditis.

Cardiac sarcoidosis or giant cell myocarditis is characterized by the presence of multinucleated giant cells, often associated with acute myocarditis in the myocardium. Since untreated giant cell myocarditides are in most cases fatal, this form of acute myocarditides has to be determined with precision, which has hitherto only been possible by histological assessment of paraffin sections. However, detection of multinucleated giant cells is extremely rare. In suspected cases, it is frequently necessary to dissect 10 or more myocardial biopsies for histological confirmation, something which almost none of the cardiological centers worldwide is prepared to do.

The microRNAs identified, whose expression is modified in such a myocarditis with myocardial giant cells, are listed in Table 8 below. With regard to the scores and significance values, reference is made to the comments on Examples 1 and 2.

TABLE 8
Expression modifications of microRNAs in cardiac
sarcoidosis/giant cell myocarditis.

| SEQ ID | | Regulation | Significance | |
|---|---|---|---|---|
| NO | microRNA | ↑↓ | value | Score |
| 111 | hsa-miR-139-3p | up | 5 | 3 |
| 112 | hsa-miR-210-3p | up | 3 | 1 |
| 113 | hsa-miR-296-5p | up | 3 | 1 |
| 114 | hsa-miR-486-3p | up | 5 | 3 |
| 115 | hsa-miR-500a-5p | up | 3 | 1 |
| 116 | hsa-miR-542-3p | up | 3 | 1 |

In one variant, the invention relates to the use of the microRNAs having a score of 1 and/or 2 and/or a significance value of 1 and/or 2 in Table 8 individually or in any combination with one another as markers for identifying giant cell myocarditis.

### Example 6

### Diagnosis of Erythrovirus-Induced Cardiomyopathy Using a Blood Serum/Blood Plasma Sample

The procedure of Example 1 was adopted, except that the blood serum/blood plasma samples were taken from patients suffering from erythrovirus-induced cardiomyopathy.

The erythrovirus causing such a cardiomyopathy occurs as two subtypes, genotype 1 being known as parvovirus B19 or erythrovirus B19. However, in myocardium infections the parvovirus plays only a minor role, so that presently the erythrovirus is not to be differentiated into its genotypes.

The microRNAs identified, whose expression is modified in such erythrovirus-induced cardiomyopathies, are listed in Table 9 below. With regard to the scores and significance values, reference is made to the comments on Examples 1 and 2.

TABLE 9
Expression modifications of microRNAs
in erythrovirus-induced cardiomyopathy.

| SEQ ID | | Regulation | Significance | |
|---|---|---|---|---|
| NO | microRNA | ↑↓ | value | Score |
| 117 | hsa-miR-103b | up | 3 | 1 |
| 118 | hsa-miR-125a-5p | down | 3 | 1 |
| 119 | hsa-miR-1271-5p | down | 3 | 1 |
| 120 | hsa-miR-1274B | down | 6 | 4 |
| 121 | hsa-miR-138-l-3p | up | 3 | 1 |
| 122 | hsa-miR-146b-3p | up | 5 | 3 |
| 123 | hsa-miR-190a-5p | up | 4 | 1 |
| 124 | hsa-miR-199a-5p | up | 5 | 3 |
| 125 | hsa-miR-26b-3p | down | 3 | 1 |
| 126 | hsa-miR-320c | up | 5 | 3 |
| 127 | hsa-miR-3617-5p | up | 3 | 1 |
| 128 | hsa-miR-362-5p | up | 5 | 3 |
| 129 | hsa-miR-3663-3p | down | 3 | 1 |
| 130 | hsa-miR-367-3p | down | 3 | 1 |
| 131 | hsa-miR-3922-5p | up | 5 | 2 |
| 132 | hsa-miR-424-5p | up | 3 | 1 |
| 133 | hsa-miR-432-5p | down | 3 | 1 |
| 134 | hsa-miR-4429 | up | 5 | 3 |
| 135 | hsa-miR-4698 | up | 3 | 1 |
| 136 | hsa-miR-4726-3p | up | 3 | 1 |
| 137 | hsa-miR-4743-5p | up | 3 | 1 |
| 138 | hsa-miR-497-5p | up | 3 | 1 |
| 139 | hsa-miR-502-3p | down | 3 | 1 |
| 140 | hsa-miR-520d-3p | down | 3 | 1 |
| 141 | hsa-miR-520f-3p | up | 5 | 4 |
| 142 | hsa-miR-597-5p | up | 5 | 3 |
| 143 | hsa-miR-625-3p | down | 3 | 1 |
| 144 | hsa-miR-628-5p | up | 5 | 3 |
| 145 | hsa-miR-629-5p | up | 3 | 1 |
| 146 | hsa-miR-758-3p | up | 3 | 1 |
| 147 | mmu-miR-374-5p | up | 6 | 3 |

In one variant, the invention relates to the use of the microRNAs having a score of 1 and/or 2 and/or a significance value of 1 and/or 2 in Table 9 individually or in any combination with one another as marker for identifying an erythrovirus-induced cardiomyopathy.

### Example 7

### Diagnosis of HHV6-Induced Cardiomyopathy Using a Blood Serum/Blood Plasma Sample

The procedure of Example 1 was adopted, except that the blood cell samples were taken from patients suffering from HHV6-induced cardiomyopathy. This includes patients suffering from ciHHV6-induced cardiomyopathy.

ciHHV6 refers to a specific form of human herpes virus 6. This virus inserts itself into the chromosomes of the host; this is referred to as chromosomal integration of HHV6 (ciHHV6).

The microRNAs identified whose expression is modified in such ciHHV6-induced cardiomyopathies are listed in Table 10 below.

Table 11 lists those identified microRNAs which are changed in a myocardial HHV6 infection. With regard to the scores and significance values, reference is made to the comments on Examples 1 and 2.

TABLE 10
Expression modifications of microRNAs in ciHHV6-induced
cardiomyopathy in a blood serum/blood plasma sample.

| SEQ ID | | Regulation | Significance | |
|---|---|---|---|---|
| NO | microRNA | ↑↓ | value | Score |
| 148 | hsa-let-7e-5p | up | 4 | 2 |
| 149 | hsa-miR-139-3p | up | 5 | 3 |
| 150 | hsa-miR-141-3p | up | 3 | 1 |
| 151 | hsa-miR-146b-3p | up | 5 | 3 |
| 152 | hsa-miR-181c-5p | up | 3 | 1 |
| 153 | hsa-miR-3156-5p | down | 3 | 1 |
| 154 | hsa-miR-362-5p | up | 5 | 3 |
| 155 | hsa-miR-411-5p | down | 4 | 3 |
| 156 | hsa-miR-422a | up | 4 | 4 |
| 157 | hsa-miR-4535 | down | 4 | 3 |
| 158 | hsa-miR-485-3p | up | 5 | 3 |
| 159 | hsa-miR-486-3p | up | 5 | 3 |
| 160 | hsa-miR-487b-3p | up | 5 | 3 |
| 161 | hsa-miR-494-3p | up | 5 | 3 |
| 162 | hsa-miR-511-5p | down | 3 | 1 |
| 163 | hsa-miR-520f-3p | down | 5 | 4 |
| 164 | hsa-miR-548c-3p | up | 5 | 3 |
| 165 | hsa-miR-548c-5p | up | 6 | 3 |
| 166 | hsa-miR-590-5p | down | 5 | 3 |
| 167 | hsa-miR-671-3p | up | 5 | 3 |
| 168 | hsa-miR-886-3p | up | 5 | 3 |
| 169 | hsa-miR-889-3p | up | 5 | 3 |
| 170 | hsa-miR-92a-3p | down | 6 | 3 |

TABLE 11
Expression modifications of microRNAs in HHV6-induced
cardiomyopathy in a blood serum/blood plasma sample.

| SEQ ID | | Regulation | Significance | |
|---|---|---|---|---|
| NO | microRNA | ↑↓ | value | Score |
| 171 | hsa-miR-192-5p | down | 3 | 1 |
| 172 | hsa-miR-29a-3p | down | 3 | 1 |
| 173 | hsa-miR-30c-5p | down | 3 | 1 |
| 174 | hsa-miR-483-5p | down | 5 | 3 |
| 175 | mmu-miR-374-5p | up | 6 | 3 |

In one variant, the invention relates to the use of the microRNAs having a score of 1 and/or 2 and/or a significance value of 1 and/or 2 in Tables 10 and 11 individually or in any combination with one another as marker for identifying HHV6-induced cardiomyopathy, in particular cardiomyopathy of the ciHHV6 form.

In one variant, the invention relates to the use of the microRNAs having a score of 1 and/or 2 and/or a significance value of 1 and/or 2 individually or in any combination with one another.

1. An in-vitro or in-vivo method for differentiating between patients requiring, for the diagnostic identification of a myocardial disorder, myocardial biopsy, and patients not requiring myocardial biopsy, and for the optional differential diagnostic differentiation between individual non-ischemic cardiomyopathies or storage diseases of the heart, wherein:
a blood serum/blood plasma sample of a patient is analyzed using a diagnostic profile comprising at least 2 microRNAs or synthetic nucleic acids having identical or complementary sequences, selected from the group consisting of the following microRNAs:
hsa-let-7f-5p (SEQ ID NO: 10), hsa-miR-103a-3p (SEQ ID NO: 11), hsa-miR-197-3p (SEQ ID NO: 12), hsa-miR-215-5p (SEQ ID NO: 13), hsa-miR-223-3p (SEQ ID NO: 14), hsa-miR-23a-3p (SEQ ID NO: 15), hsa-miR-330-3p (SEQ ID NO: 16), hsa-miR-337-5p (SEQ ID NO: 17), hsa-miR-339-3p (SEQ ID NO: 18), hsa-miR-339-5p (SEQ ID NO: 19), hsa-miR-411-5p (SEQ ID NO: 20), hsa-miR-454-3p (SEQ ID NO: 21), hsa-miR-485-3p (SEQ ID NO: 22), hsa-miR-487b-3p (SEQ ID NO: 23), hsa-miR-494-3p (SEQ ID NO: 24), hsa-miR-505-3p (SEQ ID NO: 25), hsa-miR-628-5p (SEQ ID NO: 26), hsa-miR-671-3p (SEQ ID NO: 27), hsa-miR-744-5p (SEQ ID NO: 28), hsa-miR-889-3p (SEQ ID NO: 29), mmu-miR-495-3p (SEQ ID NO: 30).

2. The in-vitro or in-vivo method as claimed in claim 1, wherein:
the diagnostic profile for identifying an adenovirus-induced cardiomyopathy additionally comprises at least 3 microRNAs or synthetic nucleic acids having identical or complementary sequences, selected from the group consisting of the following microRNAs:
hsa-miR-1274B (SEQ ID NO: 31), hsa-miR-136-3p (SEQ ID NO: 32), hsa-miR-187-3p (SEQ ID NO: 33), hsa-miR-199a-5p (SEQ ID NO: 34), hsa-miR-215-5p (SEQ ID NO: 35), hsa-miR-422a (SEQ ID NO: 36), hsa-miR-4449 (SEQ ID NO: 37), hsa-miR-4674 (SEQ ID NO: 38), hsa-miR-5096 (SEQ ID NO: 39), hsa-miR-548c-3p (SEQ ID NO: 40), hsa-miR-548c-5p (SEQ ID NO: 41), hsa-miR-650 (SEQ ID NO: 42).

3. The in-vitro or in-vivo method as claimed in claim 1 wherein:
the diagnostic profile for identifying an enterovirus-(coxsackievirus-)induced cardiomyopathy additionally comprises at least 3 microRNAs or synthetic nucleic acids having identical or complementary sequences, selected from the group consisting of the following microRNAs:
hsa-miR-1180-3p (SEQ ID NO: 43), hsa-miR-1-3p (SEQ ID NO: 44), hsa-miR-140-5p (SEQ ID NO: 45), hsa-miR-150-3p (SEQ ID NO: 46), hsa-miR-16-5p (SEQ ID NO: 47), hsa-miR-215-5p (SEQ ID NO: 48), hsa-miR-25-3p (SEQ ID NO: 49), hsa-miR-30a-5p (SEQ ID NO: 50), hsa-miR-30e-5p (SEQ ID NO: 51), hsa-miR-3138 (SEQ ID NO: 52), hsa-miR-3622b-5p (SEQ ID NO: 53), hsa-miR-3922-5p (SEQ ID NO: 54), hsa-miR-422a (SEQ ID NO: 55), hsa-miR-4274 (SEQ ID NO: 56), hsa-miR-4303 (SEQ ID NO: 57), hsa-miR-4419b (SEQ ID NO: 58), hsa-miR-4429 (SEQ ID NO: 59), hsa-miR-4467 (SEQ ID NO: 60), hsa-miR-4651 (SEQ ID NO: 61), hsa-miR-4716-5p (SEQ ID NO: 62), hsa-miR-548b-5p (SEQ ID NO: 63), hsa-miR-548c-3p (SEQ ID NO: 64), hsa-miR-597-5p (SEQ ID NO: 65), hsa-miR-601 (SEQ ID NO: 66), hsa-miR-629-3p (SEQ ID NO: 67), hsa-miR-660-5p (SEQ ID NO: 68), hsa-miR-886-3p (SEQ ID NO: 69), hsa-miR-923 (SEQ ID NO: 70), mmu-miR-374-5p (SEQ ID NO: 71).

4. The in-vitro or in-vivo method as claimed in claim 1, wherein:
the diagnostic profile for identifying amyloidosis of the heart additionally comprises at least 3 microRNAs or synthetic nucleic acids having identical or complementary sequences, selected from the group consisting of the following microRNAs:
hsa-miR-103a-3p (SEQ ID NO: 72), hsa-miR-106b-5p (SEQ ID NO: 73), hsa-miR-126-3p (SEQ ID NO: 74), hsa-miR-126-5p (SEQ ID NO: 75), hsa-miR-1274A (SEQ ID NO: 76), hsa-miR-1274B (SEQ ID NO: 77), hsa-miR-132-3p (SEQ ID NO: 78), hsa-miR-140-3p (SEQ ID NO: 79), hsa-miR-142-3p (SEQ ID NO: 80), hsa-miR-146b-5p (SEQ ID NO: 81), hsa-miR-150-5p (SEQ ID NO: 82), hsa-miR-15b-5p (SEQ ID NO: 83), hsa-miR-181b-5p (SEQ ID NO: 84), hsa-miR-186-5p (SEQ ID NO: 85), hsa-miR-21-5p (SEQ ID NO: 86), hsa-miR-222-3p (SEQ ID NO: 87), hsa-miR-223-5p (SEQ ID NO: 88), hsa-miR-24-3p (SEQ ID NO: 89), hsa-miR-26b-5p (SEQ ID NO: 90), hsa-miR-30a-3p (SEQ ID NO: 91), hsa-miR-30d-5p (SEQ ID NO: 92), hsa-miR-320a (SEQ ID NO: 93), hsa-miR-320c (SEQ ID NO: 94), hsa-miR-328-3p (SEQ ID NO: 95), hsa-miR-375 (SEQ ID NO: 96), hsa-miR-378-5p (SEQ ID NO: 97), hsa-mir-423 (SEQ ID NO: 98), hsa-miR-4286 (SEQ ID NO: 99), hsa-miR-451a (SEQ ID NO: 100), hsa-miR-4535 (SEQ ID NO: 101), hsa-miR-483-5p (SEQ ID NO: 102), hsa-miR-518d-3p (SEQ ID NO: 103), hsa-miR-532-5p (SEQ ID NO: 104), hsa-miR-590-5p (SEQ ID NO: 105), hsa-miR-601 (SEQ ID NO: 106), hsa-miR-660-5p (SEQ ID NO: 107), hsa-miR-885-5p (SEQ ID NO: 108), hsa-miR-92a-3p (SEQ ID NO: 109), hsa-miR-99b-5p (SEQ ID NO: 110).

5. The in-vitro or in-vivo method as claimed in claim 1, wherein:
the diagnostic profile for identifying lambda amyloidosis of the heart additionally comprises at least 3 microRNAs or synthetic nucleic acids having identical or complementary sequences, selected from the group consisting of the following microRNAs:
hsa-miR-106b-5p (SEQ ID NO: 73), hsa-miR-1274A (SEQ ID NO: 76), hsa-miR-30a-3p (SEQ ID NO: 91), hsa-miR-4535 (SEQ ID NO: 101).

6. The in-vitro or in-vivo method as claimed in claim 1, wherein:
diagnostic profile for identifying ATTR amyloidosis of the heart additionally comprises at least 3 microRNAs or synthetic nucleic acids having identical or complementary sequences, selected from the group consisting of the following microRNAs:
hsa-miR-140-3p (SEQ ID NO: 79), hsa-miR-181b-5p (SEQ ID NO: 84), hsa-miR-222-3p (SEQ ID NO: 87), hsa-miR-223-5p (SEQ ID NO: 88).

7. The in-vitro or in-vivo method as claimed in claim 1, wherein:
the diagnostic profile for identifying cardiac sarcoidosis/giant cell myocarditis of the heart additionally comprises at least 3 microRNAs or synthetic nucleic acids having identical or complementary sequences, selected from the group consisting of the following microRNAs:
hsa-miR-139-3p (SEQ ID NO: 111), hsa-miR-210-3p (SEQ ID NO: 112), hsa-miR-296-5p (SEQ ID NO: 113), hsa-miR-486-3p (SEQ ID NO: 114), hsa-miR-500a-5p (SEQ ID NO: 115), hsa-miR-542-3p (SEQ ID NO: 116).

8. The in-vitro or in-vivo method as claimed in claim 1, wherein:
the diagnostic profile for identifying erythrovirus-induced cardiomyopathy additionally comprises at least 3 microRNAs or synthetic nucleic acids having identical or complementary sequences, selected from the group consisting of the following microRNAs:
hsa-miR-103b (SEQ ID NO: 117), hsa-miR-125a-5p (SEQ ID NO: 118), hsa-miR-1271-5p (SEQ ID NO: 119), hsa-miR-1274B (SEQ ID NO: 120), hsa-miR-138-1-3p (SEQ ID NO: 121), hsa-miR-146b-3p (SEQ ID NO: 122), hsa-miR-190a-5p (SEQ ID NO: 123), hsa-miR-199a-5p (SEQ ID NO: 124), hsa-miR-26b-3p (SEQ ID NO: 125), hsa-miR-320c (SEQ ID NO: 126), hsa-miR-3617-5p (SEQ ID NO: 127), hsa-miR-362-5p (SEQ ID NO: 128), hsa-miR-3663-3p (SEQ ID NO: 129), hsa-miR-367-3p (SEQ ID NO: 130), hsa-miR-3922-5p (SEQ ID NO: 131), hsa-miR-424-5p (SEQ ID NO: 132), hsa-miR-432-5p (SEQ ID NO: 133), hsa-miR-4429 (SEQ ID NO: 134), hsa-miR-4698 (SEQ ID NO: 135), hsa-miR-4726-3p (SEQ ID NO: 136), hsa-miR-4743-5p (SEQ ID NO: 137), hsa-miR-497-5p (SEQ ID NO: 138), hsa-miR-502-3p (SEQ ID NO: 139), hsa-miR-520d-3p (SEQ ID NO: 140), hsa-miR-520f-3p (SEQ ID NO: 141), hsa-miR-597-5p (SEQ ID NO: 142), hsa-miR-625-3p (SEQ ID NO: 143), hsa-miR-628-5p (SEQ ID NO: 144), hsa-miR-629-5p (SEQ ID NO: 145), hsa-miR-758-3p (SEQ ID NO: 146), mmu-miR-374-5p (SEQ ID NO: 147).

9. The in-vitro or in-vivo method as claimed in claim 1, wherein:
the diagnostic profile for identifying ciHHV6-induced cardiomyopathy additionally comprises at least 3 microRNAs or synthetic nucleic acids having identical or complementary sequences, selected from the group consisting of the following microRNAs:
hsa-let-7e-5p (SEQ ID NO: 148), hsa-miR-139-3p (SEQ ID NO: 149), hsa-miR-141-3p (SEQ ID NO: 150), hsa-miR-146b-3p (SEQ ID NO: 151), hsa-miR-181c-5p (SEQ ID NO: 152), hsa-miR-3156-5p (SEQ ID NO: 153), hsa-miR-362-5p (SEQ ID NO: 154), hsa-miR-411-5p (SEQ ID NO: 155), hsa-miR-422a (SEQ ID NO: 156), hsa-miR-4535 (SEQ ID NO: 157), hsa-miR-485-3p (SEQ ID NO: 158), hsa-miR-486-3p (SEQ ID NO: 159), hsa-miR-487b-3p (SEQ ID NO: 160), hsa-miR-494-3p (SEQ ID NO: 161), hsa-miR-511-5p (SEQ ID NO: 162), hsa-miR-520f-3p (SEQ ID NO: 163), hsa-miR-548c-3p (SEQ ID NO: 164), hsa-miR-548c-5p (SEQ ID NO: 165), hsa-miR-590-5p (SEQ ID NO: 166), hsa-miR-671-3p (SEQ ID NO: 167), hsa-miR-886-3p (SEQ ID NO: 168), hsa-miR-889-3p (SEQ ID NO: 169), hsa-miR-92a-3p (SEQ ID NO: 170).

10. The in-vitro or in-vivo method as claimed in claim 1, wherein:
the diagnostic profile for identifying HHV6-induced cardiomyopathy additionally comprises at least 3 microRNAs or synthetic nucleic acids having identical or complementary sequences, selected from the group consisting of the following microRNAs:
hsa-miR-192-5p (SEQ ID NO: 171), hsa-miR-29a-3p (SEQ ID NO: 172), hsa-miR-30c-5p (SEQ ID NO: 173), hsa-miR-483-5p (SEQ ID NO: 174), mmu-miR-374-5p (SEQ ID NO: 175).

11. The in-vitro or in-vivo method as claimed in claim 1, wherein:
the non-ischemic cardiomyopathies or storage diseases of the heart are selected from the group comprising adenovirus-induced cardiomyopathy, enterovirus-(coxsackievirus-)induced cardiomyopathy, ciHHv6/HHV6-virus-induced cardiomyopathy, erythrovirus-induced cardiomyopathy, myocarditis with presence of myocardial giant cells, cardiac sarcoidosis, amyloidosis of the heart.

12. The in-vitro or in-vivo method as claimed in claim 1, wherein the following steps performed:
providing a sample obtained from a patient suspected of suffering from non-ischemic cardiomyopathy or a storage disease of the heart,
bringing the sample into contact with at least two probes each comprising a sequence corresponding to the sequences of the microRNAs from at least one of the groups as set forth in any of claims 1 to 0 or complementary to these, under conditions allowing hybridization between microRNAs contained in the sample and the probes,
determining hybridization between microRNAs of the sample and the at least one probe and
confirming the presence of a microRNA in the sample using the hybridization result of the previous step.

13. A diagnostic system for differentiation between patients requiring myocardial biopsy for diagnostic identification of a myocardial disorder and patients not requiring myocardial biopsy, and for the optional differential diagnostic differentiation between individual non-ischemic cardiomyopathies or storage diseases of the heart, characterized in that it comprises comprising:
at least two probes each comprising a sequence corresponding to a sequence of a microRNA from the group consisting of the following microRNAs or complementary to this:
hsa-miR-487b-3p (SEQ ID NO: 23), hsa-miR-494-3p (SEQ ID NO: 24), hsa-miR-889-3p (SEQ ID NO: 29), mmu-miR-495-3p (SEQ ID NO: 30).

14. The diagnostic system as claimed in claim 0, further comprising:
at least three probes each comprising a sequence corresponding to a sequence of a microRNA from the group consisting of the following microRNAs or complementary to this:
hsa-miR-1274B (SEQ ID NO: 31), hsa-miR-4449 (SEQ ID NO: 37), hsa-miR-4674 (SEQ ID NO: 38), hsa-miR-5096 (SEQ ID NO: 39), hsa-miR-548c-5p (SEQ ID NO: 41), hsa-miR-1180-3p (SEQ ID NO: 43), hsa-miR-1-3p (SEQ ID NO: 44), hsa-miR-16-5p (SEQ ID NO: 47), hsa-miR-3138 (SEQ ID NO: 52), hsa-miR-3622b-5p (SEQ ID NO: 53), hsa-miR-3922-5p (SEQ ID NO: 54), hsa-miR-4274 (SEQ ID NO: 56), hsa-miR-4303 (SEQ ID NO: 57), hsa-miR-4419b (SEQ ID NO: 58), hsa-miR-4429 (SEQ ID NO: 59), hsa-miR-4467 (SEQ ID NO: 60), hsa-miR-4651 (SEQ ID NO: 61), hsa-miR-4716-5p (SEQ ID NO: 62), hsa-miR-548b-5p (SEQ ID NO: 63), hsa-miR-597-5p (SEQ ID NO: 65), hsa-miR-923 (SEQ ID NO: 70), mmu-miR-374-5p (SEQ ID NO: 71), miR-1274A (SEQ ID NO: 76), hsa-miR-1274B (SEQ ID NO: 77), hsa-miR-223-5p (SEQ ID NO: 88), hsa-miR-328-3p (SEQ ID NO: 95), hsa-miR-378-5p (SEQ ID NO: 97), hsa-miR-423 (SEQ ID NO: 98), hsa-miR-4286 (SEQ ID NO: 99), hsa-miR-4535 (SEQ ID NO: 101), hsa-miR-210-3p (SEQ ID NO: 112), hsa-miR-103b (SEQ ID NO: 117), hsa-miR-1274B (SEQ ID NO: 120), hsa-miR-138-1-3p (SEQ ID NO: 121), hsa-miR-146b-3p (SEQ ID NO: 122), hsa-miR-190a-5p (SEQ ID NO: 123), hsa-miR-26b-3p (SEQ ID NO: 125), hsa-miR-3617-5p (SEQ ID NO: 127), hsa-miR-3663-3p (SEQ ID NO: 129), hsa-miR-3922-5p (SEQ ID NO: 131), hsa-miR-424-5p (SEQ ID NO: 132), hsa-miR-4429 (SEQ ID NO: 134), hsa-miR-4698 (SEQ ID NO: 135), hsa-miR-4726-3p (SEQ ID NO: 136), hsa-miR-4743-5p (SEQ ID NO: 137), hsa-miR-59′7-5p (SEQ ID NO: 142), hsa-miR-629-5p (SEQ ID NO: 145), hsa-miR-758-3p (SEQ ID NO: 146), mmu-miR-374-5p (SEQ ID NO: 147), hsa-miR-146b-3p (SEQ ID NO: 151), hsa-miR-3156-5p (SEQ ID NO: 153), hsa-miR-4535 (SEQ ID NO: 157), hsa-miR-487b-3p (SEQ ID NO: 160), hsa-miR-494-3p (SEQ ID NO: 161), hsa-miR-511-5p (SEQ ID NO: 162), hsa-miR-548c-5p (SEQ ID NO: 165), hsa-miR-889-3p (SEQ ID NO: 169), mmu-miR-374-5p (SEQ ID NO: 175).

15. The diagnostic system as claimed in claim 0, wherein the diagnostic system is a kit for carrying out a polymerase chain reaction and the probes are present as primers in solution or that the diagnostic system comprises a microRNA chip to which the probes have been applied.

16. A medicament for the treatment of non-ischemic cardiomyopathies or storage diseases of the heart, comprising at least one nucleic acid comprising a sequence identical to or complementary with the sequence of one of the microRNAs as set forth in claim 0.

17. The diagnostic system as claimed in claim 0, wherein the diagnostic system is a kit for carrying out a polymerase chain reaction and the probes are present as primers in solution or that the diagnostic system comprises a microRNA chip to which the probes have been applied.

18. A medicament for the treatment of non-ischemic cardiomyopathies or storage diseases of the heart, comprising at least one nucleic acid comprising a sequence identical to or complementary with the sequence of one of the microRNAs as set forth in claim 0.
